(19) United States
(12) Patent Application Publication
(10) Pub. No.: US 2021/0003556 A1
(43) Pub. Date: Jan. 7, 2021

(54) IMPROVING ANTI-PD-1 CANCER THERAPY

(71) Applicant: Mayo Foundation for Medical Education and Research, Rochester, MN (US)

(72) Inventor: Haidong Dong, Rochester, MN (US)

(21) Appl. No.: 16/979,320

(22) PCT Filed: Mar. 12, 2019
(86) PCT No.: PCT/US19/21802

§ 371 Date: Sep. 9, 2020

Related U.S. Application Data
(60) Provisional application No. 62/641,672, filed on Mar. 12, 2018.

(51) Int. Cl. G01N 33/50; A61K 38/20; A61K 31/337; A61K 31/282; A61K 39/395; C07K 16/28; A61P 35/00

(52) U.S. Cl. CPC G01N 33/505; A61K 38/2086; A61K 31/337; A61K 31/282; A61K 39/39541; A61K 2039/505; A61P 35/00; G01N 2333/7158; G01N 2333/705; G01N 2333/96436; G01N 2800/52; C07K 16/2818

(57) ABSTRACT

Materials and methods for identifying and treating cancer patients who are likely to respond to chemo-immunotherapy (CIT) and other cancer treatments are provided herein, including materials and methods for using CX3CR1 to identify PD-1 therapy-responsive CD8+ T cells that withstand the toxicity of chemotherapy during combined CIT.

### CROSS-REFERENCE TO RELATED APPLICATIONS

This application claims benefit of priority from U.S. Provisional Application Ser. No. 62/641,672, filed on Mar. 12, 2018. The disclosure of the prior application is considered part of (and is incorporated by reference in) the disclosure of this application.

### TECHNICAL FIELD

This document relates to materials and methods for identifying cancer patients who are likely to respond to chemo-immunotherapy (CIT), including materials and methods for using CX3CR1 to identify PD-1 therapy-responsive CD8⁺ T cells that withstand the toxicity of chemotherapy during combined cancer CIT.

### BACKGROUND

Immune checkpoint inhibitor (ICI) therapies targeted to programmed cell death protein-1 (PD-1)/programmed death ligand-1 (PD-L1) have achieved a durable clinical benefit in a subset of patients with cancer. Unlike chemotherapy or radiation therapy, PD-1 ICI therapy does not directly destroy tumor cells, but rather works through at least two steps: (1) blocking PD-1 signals in T cells; and (2) expanding immune effector cells capable of rejecting tumor cells. However, primary or acquired resistance to PD-1 ICI is common, and is a pressing challenge in cancer immunotherapy. Some cancer patients with tumors that progressed upon anti-PD-1 therapy have benefitted from the addition of salvage chemotherapy, even though cytotoxic chemotherapy has been viewed as toxic to immune cells. The mechanism responsible for the successful clinical outcomes of CIT is not completely understood.

### SUMMARY

This document is based, at least in part, on the discovery that a subset of tumor-reactive CD8⁺ T cells, expressing the chemokine receptor CX3CR1, endured cytotoxic chemotherapy and significantly increased in response to combined chemo-immunotherapy (paclitaxel and carboplatin with PD-1 blockade) in metastatic melanoma patients. These CX3CR1⁺CD8⁺ T cells have an effector memory phenotype and the ability to efflux chemotherapy drugs via the ABCB1 transporter. This document also is based, at least in part, on the identification of a combination and sequence of CIT that results in an increase in CX3CR1⁺CD8⁺ T cells required for mediating tumor regression. The studies described herein define a critical role for CX3CR1⁺ CD8⁺ tumor-reactive T cells in the success of CIT, promoting their development as a marker for monitoring patient responses to CIT.

This document also is based, at least in part, on the discovery that % Bim⁺ CD8⁺ T cells can be used as a molecular marker for PD-1 blockade-responsiveness. This marker, in combination with the CX3CR1⁺ CD8⁺ T cell marker, can be used not only to predict the degree to which PD-1 ICI therapy has turned a patient's immune system to reject tumors, but also to aid in identifying patients who would likely benefit from an appropriate combined therapy. For example, some patients may demonstrate responses to PD-1 blockade (with a decrease of Bim⁺ CD8⁺ T cells), but without a clinical response due to lack of sufficient effector cells (CX3CR1⁺ Granzyme CD8⁺ T cells). For such patients, continued application of PD-1 ICI may still provide the benefit of preventing CD8⁺ T cells from apoptosis mediated by high Bim expression, and also provide a window for combined therapy that can reduce tumor burden and expand effector T cells.

In a first aspect, this document features a method that includes measuring the percentage of CX3CR1⁺ cells and the percentage of Bim⁺ cells within a first population of CD8⁺ T cells obtained from a subject having a tumor, where the first population of CD8⁺ T cells was obtained prior to treatment of the subject with PD-1 blockade therapy; measuring the percentage of CX3CR1⁺ cells and the percentage of Bim⁺ cells within a second population of CD8⁺ T cells obtained from the subject, where the second population of CD8⁺ T cells was obtained after treatment of the subject with PD-1 blockade therapy; identifying the subject as having a percentage of CX3CR1⁺ cells within the second population that is increased by at least a predetermined CX3CR1⁺ threshold relative to the percentage of CX3CR1⁺ cells within the first population and as having a percentage of Bim⁺ cells within the second population that is decreased by at least a predetermined Bim⁺ threshold relative to the percentage of Bim⁺ cells within the first population; and treating the subject with a therapy to increase tumor immunogenicity. The predetermined CX3CR1⁺ threshold can be an increase of at least 80%, and the predetermined Bim⁺ threshold can be a decrease of at least 20%. The first and second populations of CD8⁺ T cells can be obtained from the peripheral blood of the subject, or from the tumor. The subject can be a human. The tumor can contain metastatic melanoma cells, gastrointestinal cancer cells, genitourinary cancer cells, non-small lung cancer cells, or breast cancer cells. The measuring can include using flow cytometry, time of flight mass cytometry (cyToF), immunohistochemistry (IHC), multiplex immunofluorescence imaging analysis, or single cell or sorted cell-RNA-sequencing analysis. The therapy to increase tumor immunogenicity can include radiation. The method can include measuring the percentage of CX3CR1⁺ Granzyme B⁺ cells within the first and second populations.

In another aspect, this document features a method that includes measuring the percentage of CX3CR1⁺ cells and the percentage of Bim⁺ cells within a first population of CD8⁺ T cells obtained from a subject having a tumor, where the first population of CD8⁺ T cells was obtained prior to treatment of the subject with PD-1 blockade therapy; measuring the percentage of CX3CR1⁺ cells and the percentage of Bim⁺ cells within a second population of CD8⁺ T cells obtained from the subject, where the second population of CD8⁺ T cells was obtained after treatment of the subject with PD-1 blockade therapy; identifying the subject as having a percentage of CX3CR1⁺ cells within the second population that is increased by less than a predetermined CX3CR1⁺ threshold relative to the percentage of CX3CR1⁺ cells within the first population and as having a percentage of Bim⁺ cells within the second population that is decreased by at least a predetermined Bim⁺ threshold relative to the percentage of Bim⁺ cells within the first population; and treating the subject with cytokine therapy combined with PD-1 blockade therapy. The predetermined CX3CR1⁺ threshold can be an increase of at least 80%, and the predetermined Bim⁺ threshold can be a decrease of at least 20%. The first and second populations of CD8⁺ T cells can be obtained from the peripheral blood of the subject, or from the tumor. The subject can be a human. The tumor can contain metastatic melanoma cells, gastrointestinal cancer cells, genitourinary cancer cells, non-small lung cancer cells, or breast cancer cells. The measuring can include using flow cytometry, cyToF, IHC, multiplex immunofluorescence imaging analysis, or single cell or sorted cell-RNA-sequencing analysis. The cytokine therapy can include treatment with IL-15. The method can include measuring the percentage of CX3CR1⁺ Granzyme B⁺ cells within the first and second populations.

In another aspect, this document features a method that includes measuring the percentage of CX3CR1⁺ cells and the percentage of Bim⁺ cells within a first population of CD8⁺ T cells obtained from a subject having a tumor, where the first population of CD8⁺ T cells was obtained prior to treatment of the subject with PD-1 blockade therapy; measuring the percentage of CX3CR1⁺ cells and the percentage of Bim⁺ cells within a second population of CD8⁺ T cells obtained from the subject, where the second population of CD8⁺ T cells was obtained after treatment of the subject with PD-1 blockade therapy; identifying the subject as having a percentage of CX3CR1⁺ cells within the second population that is increased by at least a predetermined CX3CR1⁺ threshold relative to the percentage of CX3CR1⁺ cells within the first population and as having a percentage of Bim⁺ cells within the second population that is increased, is unchanged, or is decreased by less than a predetermined Bim⁺ threshold relative to the percentage of Bim⁺ cells within the first population; and treating the subject with combined CIT. The predetermined CX3CR1⁺ threshold can be an increase of at least 80%, and the predetermined Bim⁺ threshold can be a decrease of at least 20%. The first and second populations of CD8⁺ T cells can be obtained from the peripheral blood of the subject, or from the tumor. The subject can be a human. The tumor can contain metastatic melanoma cells, gastrointestinal cancer cells, genitourinary cancer cells, non-small lung cancer cells, or breast cancer cells. The measuring can include using flow cytometry, cyToF, IHC, multiplex immunofluorescence imaging analysis, or single cell or sorted cell-RNA-sequencing analysis. The CIT can include treatment with paclitaxel, carboplatin, and anti-PD-1 or anti-PD-L1 therapy. The method can include measuring the percentage of CX3CR1⁺ Granzyme B⁺ cells within the first and second populations.

In another aspect, this document features a method that includes measuring the percentage of CX3CR1⁺ cells and the percentage of Bim⁺ cells within a first population of CD8⁺ T cells obtained from a subject having a tumor, where the first population of CD8⁺ T cells was obtained prior to treatment of the subject with PD-1 blockade therapy; measuring the percentage of CX3CR1⁺ cells and the percentage of Bim⁺ cells within a second population of CD8⁺ T cells obtained from the subject, where the second population of CD8⁺ T cells was obtained after treatment of the subject with PD-1 blockade therapy; identifying the subject as having a percentage of CX3CR1⁺ cells within the second population that is increased by less than a predetermined CX3CR1⁺ threshold relative to the percentage of CX3CR1⁺ cells within the first population and as having a percentage of Bim⁺ cells within the second population that is increased, is unchanged, or is decreased by less than a predetermined Bim⁺ threshold relative to the percentage of Bim⁺ cells within the first population; and treating the subject with an ICI therapy other than PD-1 blockade, optionally in combination with chemotherapy. The predetermined CX3CR1⁺ threshold can be an increase of at least 80%, and the predetermined Bim⁺ threshold can be a decrease of at least 20%. The first and second populations of CD8⁺ T cells can be obtained from the peripheral blood of the subject, or from the tumor. The subject can be a human. The tumor can contain metastatic melanoma cells, gastrointestinal cancer cells, genitourinary cancer cells, non-small lung cancer cells, or breast cancer cells. The measuring can include using flow cytometry, cyToF, IHC, multiplex immunofluorescence imaging analysis, or single cell or sorted cell-RNA-sequencing analysis. The ICI therapy can include treatment with anti-TIGIT and/or anti-Tim 3. The method can include measuring the percentage of CX3CR1⁺ Granzyme B⁺ cells within the first and second populations.

In another aspect, this document features a method that includes measuring the percentage of CX3CR1⁺ cells within a population of CD8⁺ T cells obtained from a subject having a tumor, identifying the subject as being likely to respond to combined CIT when the percentage of CX3CR1⁺ cells within the population is increased relative to a corresponding control percentage of CX3CR1⁺ cells, and administering the CIT to the subject. The population of CD8⁺ T cells can be obtained from the peripheral blood of the subject, or from the tumor. The method can include obtaining the population of CD8⁺ T cells before treatment of the subject with the CIT, after treatment of the subject with the CIT, after treatment of the subject with chemotherapy (e.g., paclitaxel, carboplatin, or a combination thereof) or after treatment of the subject with ICI therapy (anti-PD-1 or anti-PD-L1 therapy). The CIT can include paclitaxel, carboplatin, and anti-PD-1 or anti-PD-L1 therapy. The subject can be a human. The tumor can contain metastatic melanoma cells, gastrointestinal cancer cells, genitourinary cancer cells, non-small lung cancer cells, or breast cancer cells. The measuring can include using flow cytometry, cyToF, IHC, multiplex immunofluorescence imaging analysis, or single cell or sorted cell-RNA-sequencing analysis. The corresponding control percentage can be the percentage of CX3CR1⁺ cells in a population of CD8⁺ T cells obtained from the subject at baseline. The method can include measuring the percentage of CX3CR1⁺ Granzyme B⁺ cells within the population, and identifying the subject as being likely to respond to the CIT when the percentage of CX3CR1⁺ Granzyme B⁺ cells within the population is increased relative to a corresponding control percentage of CX3CR1⁺ Granzyme B⁺ cells (e.g., the percentage of CX3CR1⁺ Granzyme B⁺ cells in a population of CD8⁺ T cells obtained from the subject at baseline). The method can further include measuring the percentage of Bim⁺ CD8⁺ T cells within the population, and identifying the subject as being likely to respond to CIT when the percentage of Bim⁺ CD8⁺ T cells within the population is decreased relative to a corresponding control percentage of Bim⁺ CD8⁺ T cells (e.g., the percentage of Bim⁺ cells in a population of CD8⁺ T cells obtained from the subject at baseline).

In another aspect, this document features a method that includes measuring the percentage of CX3CR1⁺ cells within a first population of CD8⁺ T cells obtained from a subject having a tumor, wherein the first population was obtained from the tumor prior to CIT, administering the CIT to the subject, measuring the percentage of CX3CR1⁺ cells within a second population of CD8⁺ T cells obtained from the subject, wherein the second population was obtained from the tumor after CIT, and identifying the subject as being responsive to the CIT when the percentage of CX3CR1⁺ cells within the second population is increased relative to the percentage of CX3CR1+ cells within the first population. The first and second populations of CD8⁺ T cells can be obtained from the peripheral blood of the subject, or from the tumor. The method can include obtaining the first population of CD8⁺ T cells after treatment of the subject with chemotherapy (e.g., paclitaxel, carboplatin, or a combination thereof), or after treatment of the subject with ICI therapy (e.g., anti-PD-1 or anti-PD-L1 therapy). The CIT can include paclitaxel, carboplatin, and anti-PD-1 or anti-PD-L1 therapy. The subject can be a human. The tumor can contain metastatic melanoma cells, gastrointestinal cancer cells, genitourinary cancer cells, non-small lung cancer cells, or breast cancer cells. The measuring can include using flow cytometry, cyToF, IHC, multiplex immunofluorescence imaging analysis, or single cell or sorted cell-RNA-sequencing analysis. The method can include measuring the percentage of CX3CR1⁺ Granzyme B⁺ cells within the first and second populations, and identifying the subject as being responsive to the CIT when the percentage of CX3CR1⁺ Granzyme B⁺ cells within the second population is increased relative the percentage of CX3CR1⁺ Granzyme B⁺ cells within the first population. The method can further include measuring the percentage of Bim⁺ CD8⁺ T cells within the first and second populations, and identifying the subject as being responsive to the CIT when the percentage of Bim⁺ CD8⁺ T cells within the second population is decreased relative to the percentage of Bim⁺ CD8⁺ T cells within the first population.

In yet another aspect, this document features a method that includes obtaining a population of CD8⁺ T cells from a subject having a tumor, measuring the percentage of CX3CR1⁺ Granzyme B⁺ cells within the population of CD8⁺ T cells, identifying the subject as being likely to respond to CIT when the percentage of CX3CR1⁺ Granzyme B⁺ cells within the population is increased relative to a corresponding control percentage; and administering the CIT to the subject. The population of CD8⁺ T cells can be obtained from the peripheral blood of the subject, or from the tumor. The method can include obtaining the population of CD8⁺ T cells before treatment of the subject with the CIT, after treatment of the subject with the CIT, after treatment of the subject with chemotherapy (e.g., paclitaxel, carboplatin, or a combination thereof), or after treatment of the subject with ICI therapy (e.g., anti-PD-1 or anti-PD-L1 therapy). The CIT can include paclitaxel, carboplatin, and anti-PD-1 or anti-PD-L1 therapy. The subject can be a human. The tumor can contain metastatic melanoma cells, gastrointestinal cancer cells, genitourinary cancer cells, non-small lung cancer cells, or breast cancer cells. The measuring can include using flow cytometry, cyToF, IHC, multiplex immunofluorescence imaging analysis, or single cell or sorted cell-RNA-sequencing analysis. The corresponding control percentage can be the percentage of CX3CR1⁺ Granzyme B⁺ cells in a population of CD8⁺ T cells obtained from the subject at baseline. The method can further include measuring the percentage of Bim⁺ CD8⁺ T cells within the population, and identifying the subject as being likely to respond to CIT when the percentage of Bim⁺ CD8⁺ T cells within the population is decreased relative to a corresponding control percentage of Bim⁺ CD8⁺ T cells (e.g., the percentage of Bim⁺ cells in a population of CD8⁺ T cells obtained from the subject at baseline).

In another aspect, this document features a method that includes measuring the percentage of CX3CR1⁺ Granzyme B⁺ cells within a first population of CD8⁺ T cells obtained from a subject having a tumor, wherein the first population was obtained from the tumor prior to CIT, administering the CIT to the subject, measuring the percentage of CX3CR1⁺ Granzyme B⁺ cells within a second population of CD8⁺ T cells obtained from the subject, wherein the second population was obtained from the tumor after CIT, and identifying the subject as being responsive to the CIT when the percentage of CX3CR1⁺ Granzyme B⁺ cells within the second population is increased relative to the percentage of CX3CR1⁺ Granzyme B⁺ cells within the first population. The first and second populations of CD8⁺ T cells can be obtained from the peripheral blood of the subject or from the tumor. The method can include obtaining the first population of CD8⁺ T cells after treatment of the subject with chemotherapy (e.g., paclitaxel, carboplatin, or a combination thereof), or after treatment of the subject with ICI therapy (e.g., anti-PD-1 or anti-PD-L1 therapy). The CIT can include paclitaxel, carboplatin, and anti-PD-1 or anti-PD-L1 therapy. The subject can be a human. The tumor can contain metastatic melanoma cells, gastrointestinal cancer cells, genitourinary cancer cells, non-small lung cancer cells, or breast cancer cells. The measuring can include using flow cytometry, cyToF, IHC, multiplex immunofluorescence imaging analysis, or single cell or sorted cell-RNA-sequencing analysis. The method can include measuring the percentage of CX3CR1⁺ Granzyme B⁺ cells within the first and second populations, and identifying the subject as being responsive to the CIT when the percentage of CX3CR1⁺ Granzyme B⁺ cells within the second population is increased relative the percentage of CX3CR1⁺ Granzyme cells within the first population. The method can further include measuring the percentage of Bim⁺ CD8⁺ T cells within the first and second populations, and identifying the subject as being responsive to the CIT when the percentage of Bim⁺ CD8⁺ T cells within the second population is decreased relative to the percentage of Bim⁺ CD8⁺ T cells within the first population.

This document also features a method for expanding a population of CX3CR1⁺ CD8⁺ T cells, where the method includes obtaining a population of CX3CR1⁺ CD8⁺ T cells from a subject, contacting the population with interleukin-15 (IL-15), and determining that the population of CX3CR1⁺ CD8⁺ T cells has expanded. The population of CD8⁺ T cells can be obtained from the peripheral blood of the subject, or from a tumor in the subject. The tumor can contain metastatic melanoma cells, gastrointestinal cancer cells, genitourinary cancer cells, non-small lung cancer cells, or breast cancer cells. The determining can include using flow cytometry, cyToF, IHC, multiplex immunofluorescence imaging analysis, or single cell or sorted cell-RNA-sequencing analysis to assess the number of CX3CR1⁺ CD8⁺ T cells. The method can further include administering at least a portion of the expanded CX3CR1⁺ CD8⁺ T cell population to the subject.

In addition, this document features a method that includes measuring the percentage of CX3CR1⁺ cells within a first population of CD8⁺ T cells obtained from a subject having a tumor, administering IL-15 to the subject, measuring the percentage of CX3CR1⁺ cells within a second population of CD8⁺ T cells obtained from the subject after the IL-15 administration, and determining that the percentage of CX3CR1⁺ cells within the second population is increased relative to the percentage in the first population. The first and second populations of CD8⁺ T cells can be within a peripheral blood sample from the subject, or from a tumor within the subject. The tumor can contain metastatic melanoma cells, gastrointestinal cancer cells, genitourinary cancer cells, non-small lung cancer cells, or breast cancer cells. The measuring can include using flow cytometry, cyToF, IHC, multiplex immunofluorescence imaging analysis, or single cell or sorted cell-RNA-sequencing analysis to assess the number of CX3CR1⁺ CD8⁺ T cells.

In still another aspect, this document features a method for identifying a subject in need of treatment modification. The method can include measuring the percentage of CX3CR1⁺ cells and the percentage of Bim⁺ cells within a first population of CD8⁺ T cells obtained from a subject having a tumor, wherein the first population of CD8⁺ T cells was obtained prior to treatment of the subject with PD-1 blockade therapy; measuring the percentage of CX3CR1⁺ cells and the percentage of Bim⁺ cells within a second population of CD8⁺ T cells obtained from the subject, wherein the second population of CD8⁺ T cells was obtained after treatment of the subject with PD-1 blockade therapy; identifying the subject as having a percentage of CX3CR1⁺ cells within the second population that is increased by at least a predetermined CX3CR1⁺ threshold relative to the percentage of CX3CR1⁺ cells within the first population, or is increased by less than the predetermined CX3CR1⁺ threshold relative to the percentage of CX3CR1⁺ cells within the first population; and identifying the subject as having a percentage of Bim⁺ cells within the second population that is decreased by at least a predetermined Bim⁺ threshold relative to the percentage of Bim⁺ cells within the first population, or is increased, unchanged, or decreased by less than the predetermined Bim⁺ threshold relative to the percentage of Bim⁺ cells within the first population, thereby identifying the subject as being in need of a therapy other than or in addition to the PD-1 blockade therapy. The method can include identifying the subject as having a percentage of CX3CR1⁺ cells within the second population that is increased by at least the predetermined CX3CR1⁺ threshold relative to the percentage of CX3CR1⁺ cells within the first population and as having a percentage of Bim⁺ cells within the second population that is decreased by at least the predetermined Bim⁺ threshold relative to the percentage of Bim⁺ cells within the first population, thereby identifying the subject as being in need of a therapy to increase tumor immunogenicity (e.g., a therapy that includes radiation). The method can include identifying the subject as having a percentage of CX3CR1⁺ cells within the second population that is increased by less than the predetermined CX3CR1⁺ threshold relative to the percentage of CX3CR1⁺ cells within the first population and as having a percentage of Bim⁺ cells within the second population that is decreased by at least the predetermined Bim⁺ threshold relative to the percentage of Bim⁺ cells within the first population, thereby identifying the subject as being in need of cytokine therapy (e.g., treatment with IL-15) combined with PD-1 blockade therapy. The method can include identifying the subject as having a percentage of CX3CR1⁺ cells within the second population that is increased by at least the predetermined CX3CR1⁺ threshold relative to the percentage of CX3CR1⁺ cells within the first population and as having a percentage of Bim⁺ cells within the second population that is increased, is unchanged, or is decreased by less than the predetermined Bim⁺ threshold relative to the percentage of Bim⁺ cells within the first population, thereby identifying the subject as being in need of CIT (e.g., treatment with paclitaxel, carboplatin, and anti-PD-1 or anti-PD-L1 therapy). The method can include identifying the subject as having a percentage of CX3CR1⁺ cells within the second population that is increased by less than the predetermined CX3CR1⁺ threshold relative to the percentage of CX3CR1⁺ cells within the first population and as having a percentage of Bim⁺ cells within the second population that is increased, is unchanged, or is decreased by less than the predetermined Bim⁺ threshold relative to the percentage of Bim⁺ cells within the first population, thereby identifying the subject as being in need of an ICI therapy other than PD-1 blockade (e.g., treatment with anti-TIGIT and/or anti-Tim 3), optionally in combination with chemotherapy. The predetermined CX3CR1⁺ threshold can be an increase of at least 80%. The predetermined Bim⁺ threshold can be a decrease of at least 20%. The predetermined CX3CR1⁺ threshold can be an increase of at least 80% and the predetermined Bim⁺ threshold can be a decrease of at least 20%. The first and second populations of CD8⁺ T cells can be from the peripheral blood of the subject, or can be from the tumor. The subject can be a human. The tumor can contain metastatic melanoma cells, gastrointestinal cancer cells, genitourinary cancer cells, non-small lung cancer cells, or breast cancer cells. The measuring can include using flow cytometry, cyToF, IHC, multiplex immunofluorescence imaging analysis, or single cell or sorted cell-RNA-sequencing analysis.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A to 1E illustrate increased expression of CX3CR1 in responders to PD-1 therapy. FIG. 1A is an RNA-seq graph showing that among tumor-reactive (PD-1⁺CD11a^{high}) CD8⁺ T cells in the peripheral blood of melanoma patients prior to PD-1 therapy, there is increased transcription of CX3CR1 (arrow) in responders (R, n=3) compared to non-responders (NR, n=3) at baseline prior to anti-PD-1 therapy. Data represent the average levels of CX3CR1 transcription and expression of three patients. FIG. 1B is an RNA-seq graph showing increased transcription of CX3CR1, CD122 (IL2RB), KLRG1, Perforin (PRF1), Granzyme B (GZMB) (arrows) and TCRVα5/TCRVβ4-2 (arrow heads). Data represent the average levels of transcription for three or two patients (R, n=3; NR=2) with at least 2-fold changes. FIG. 1C is a flow cytometry plot and graphed results showing PD-1 expression by CX3CR1⁺ CD11a^{high} or CX3CR1⁻ CD11a^{low} peripheral CD8⁺ T cells isolated from patients with melanoma prior to PD-1 therapy (n=12, ***P<0.01, Paired t test). FIG. 1D is a series of flow cytometry plots and graphs plotting the frequency of CX3CR1⁺ Granzyme cells among CD11a^{high} CD8⁺ T cells, showing that the CX3CR1⁺ Granzyme B⁺ cells were significantly increased in responders after anti-PD-1 therapy in melanoma patients (n=7, **P<0.05), but not at baseline prior to PD-1 therapy (NS, not significant). FIG. 1E is an image showing staining of CX3CR1⁺ Granzyme B⁺ cells (double positive staining, DP) in human melanoma tissues. One DP cell was inside the tumor bed (black arrow), and another adhered to a blood vessel, probably in the stage of extravasation (white arrow).

FIGS. 2A to 2C show patient responses to CIT with an increase of CX3CR1⁺ Granzyme B⁺ CD8⁺ T cells. FIG. 2A shows a timeline and a series of PET/CT scan images for a patient with BRAF wild-type metastatic melanoma who received previous ipilimumab adjuvant therapy and then was started on pembrolizumab single-agent (at 2 mg/kg) due to disease progression. PET/CT scan results were collected at each time point (arrows) to demonstrate the disease status. Given the disease progression while on pembrolizumab, paclitaxel, and carboplatin (white arrows) were initiated at 175 mg/m² and an AUC (area under curve) of 5 every 3 weeks for 2 cycles in combination with pembrolizumab. The patient received total of 12 cycles of pembrolizumab at the end of the follow up. FIG. 2B is a pair of flow cytometry plots showing CX3CR1 levels pre- and post-chemo, as indicated. Following the same schedule of treatment as in FIG. 2A, blood samples were collected for flow analysis of CX3CR1⁺ Granzyme B⁺ among CD11a^{high}CD8⁺ T cells. FIG. 2C is a graph plotting the frequency of CX3CR1⁺ Granzyme B⁺ among CD11a^{high}CD8⁺ T cells in responders (n=3 pre, 4 post) and non-responders (n=4) pre- and post-chemotherapy as treated for FIG. 2A. *p<0.05 between responders and non-responders to CIT. FIGS. 2D and 2E show CTL function (CD107a expression and IFN-γ production) and proliferation (Ki67 expression) of CX3CR1⁺ CD11a^{high} or CX3CR1⁻ CD11a^{low} peripheral CD8⁺ T cells isolated from responders (n=3) prior to (pre) and after (post) PD-1 therapy. FIG. 2D is a series of representative flow cytometry plots showing the CTL function of CX3CR1⁺ or CX3CR1⁻ CD8⁺ T cells from a CIT responder (n=3) after a brief ex vivo stimulation of T cells with PMA and ionomycin. FIG. 2E is a pair of graphs plotting the percent of CX3CR1⁺ and CX3CR1″ CD8⁺ T cells that were CD107a⁺ IFN-γ⁺ or Ki67⁺, as indicated.

FIGS. 3A to 311 are a series a graphs showing the efflux of chemotherapy drug by human CX3CR1⁺ CD8⁺ T cells. Purified human primary CD8⁺ T cells were loaded with Doxorubicin (1 μg/ml) for 30 minutes and then washed before further incubation for 60 minutes (FIG. 3A) or for the indicated times (FIG. 3B). The gated areas in FIG. 3A are efflux cells (Dox^{low}CX3CR1^{high}). For FIG. 3C, CD8⁺ T cells were incubated with Doxorubicin (0.5 μg/ml) for 40 hours and then stained with Annexin V to identify apoptotic cells. FIG. 3D shows expression of ABCB1 by CX3CR1⁺ or CX3CR1⁻ CD8⁺ T cells. FIG. 3E shows that the ABCB1 inhibitor PGP4008 reduced the drug efflux ability of CX3CR1⁺ CD8⁺ T cells. Cells incubated on ice after loading with drug were used as a negative control of drug efflux. FIG. 3F demonstrates that the ABCB1 inhibitor PGP4008 increased apoptosis of CX3CR1⁺ CD8⁺ T cells cultured as for FIG. 3C. The impact of the ABCB1 inhibitor on the function of human CX3CR1⁺ CD8⁺ T cells incubated with or without chemotherapy drugs (carboplatin and paclitaxel) is shown in FIG. 3G and FIG. 3H, respectively. CD8⁺ T cells were activated with anti-CD3/CD28 beads for 24 hours in the presence of DMSO (control) or PGP4008 (10 Cytotoxic T lymphocyte (CTL) function was measured as CD107a expression and IFN-γ production at the end of culture. *P<0.05; **P<0.01 (Mann-Whitney U test two-tailed). NS, not significant.

FIG. 4 is a series of flow cytometry plots and graphs showing that CX3CR1⁺ CD8⁺ T cells express ABCB1 and have efflux function. Human peripheral blood CD8⁺ T cells were loaded with 10 μg/ml of Rh123 on ice for 30 minutes and then washed with PBS and incubated for another 30 minutes at 37° C. T cell expression of ABCB1/CX3CR1 and efflux of Rh123 were analyzed by flow cytometry. The percentage of efflux of Rh123 was higher in ABCB1⁺ CX3CR1⁺ T cells than in ABCB1⁻ CX3CR1⁻ T cells. DP: double positive; DN: double negative. **P<0.01 (Mann-Whitney U test Two-tailed, n=6).

FIGS. 5A to 5E show that CX3CR1⁺ Granzyme CD8⁺ T cells are increased after CIT. Once B16F10 mouse melanoma tumors were palpable on day 7 after tumor injection, animals were randomly assigned to treatment groups. FIG. 5A illustrates that schedule of treatments. Mice were treated with intraperitoneal (i.p.) injection of anti-PD-1 and PD-L1 antibodies (at 100 μg of each antibody) and collectively indicated as anti-PD or control IgG for a total of five doses at 3-day intervals. Carboplatin (40 μg/g) and paclitaxel (10 μg/g body weight) (collectively indicated as CP) were injected i.p. either on day 7 or on day 10 after tumor injection. FIG. 5B is a graph plotting tumor growth. Data show the mean±SEM of five mice per group, **P<0.01 compared between day 7 and 10 treatment with CP plus anti-PD, ***P<0.001 compared between day 10 CP only and control or day 10 CP plus PD-1 blockade (Two-way ANOVA). FIG. 5C is a graph plotting the survival rate of treated animals as in FIG. 5B. *P<0.05 compared between control and anti-PD groups (log-rank test). FIG. 5D is a graph plotting the frequency of CX3CR1⁺ Granzyme CD8⁺ T cells measured in CD11a^{high} CD8⁺ cells isolated from tumor tissues on day 16 after tumor injection (*P<0.05, N=6 Two-way ANOVA). FIG. 5E is a graph plotting B16F10 tumor growth in wild type (WT) and PD-1 knockout (KO) mice after treatment with carboplatin and paclitaxel (CP) as in FIG. 5B on day 8 after tumor injection. The graph represents one of two independent experiments (***P<0.001, N=3-5, Two-way ANOVA).

FIGS. 6A to 6E are a series of graphs indicating that the lack of CX3CR1 abolishes the antitumor activity of CIT. CX3CR1 deficient (FIG. 6A, male; FIG. 6B, female) mice were injected with B16F10 tumor cells and then treated by i.p. injection of anti-PD-1 and PD-L1 antibody (100 μg of each antibody, collectively indicated as anti-PD) or control IgG for a total of five doses at 3-day intervals starting on day 7 after tumor injection. Carboplatin (40 μg/g) and paclitaxel (10 μg/g body weight) (collectively indicated as CP) were injected i.p. once, either on day 7 or on day 10 after tumor injection. Data show the mean±SEM of five mice per group. FIG. 6C illustrates the frequency of CD107a⁺ IFN-γ⁺ cells among CD11a^{high} CD8⁺ T cells isolated from tumor tissues decreased in CX3CR1 KO mice, compared to wild type mice. *P<0.05 (Mann-Whitney U test Two-tailed, N=5). FIG. 6D is a graph plotting tumor size after adoptive transfer of CX3CR1⁺ OT-1 CD8⁺ T cells or CX3CR1⁻ OT-1 CD8⁺ T cells. The CX3CR1⁺ OT-1 CD8⁺ T cells suppressed the growth of B16-OVA tumors. Data show the mean±SEM of five mice per group, **P<0.01 (Two-way ANOVA). Data from one of two independent experiments are shown. FIG. 6E is a Venn diagram showing three genes that were up-regulated in CX3CR1 Knockout (KO) CD8⁺ T cells as compared to wild type (WT) CD8⁺ T cells; the up-regulation of these genes was shared among three status groups (resting, 24-hour, and 48-hour activation with anti-CD3/CD28 beads in vitro).

FIG. 7 is a graph plotting CX3CR1⁺ CD8⁺ and CX3CR1⁻ CD8⁺ T cell survival during treatment with doxorubicin (Dox) in vitro. CX3CR1⁺ and CX3CR1⁻ CD8⁺ T cell subsets were incubated with Dox and then stained with annexin V. The graph shows the percentage of Dox⁺/annexin V low (live) cells in each subset of CD8⁺ T cells. *p<0.05 by unpaired t test. N=4 donors.

FIGS. 8A to 8C demonstrate expression of CD122 by human CD8⁺ T cells. FIG. 8A is a flow cytometry plot showing representative CD122 expression by CX3CR1⁺ CD8⁺ T cells. FIG. 8B is a graph plotting CD122 expression by CX3CR1⁺ and CX3CR1⁻ CD8⁺ T cells in PBMC after incubation with PHA-L for 48 hours. Data show the mean±SD (n=4 donors). **P<0.01 by Mann Whitney test. FIG. 8C is a graph plotting proliferation of CX3CR1⁺ CD8⁺ T cells treated in vitro with human IL-15 for 48 hours. Data show % Ki67⁺ cells among CX3CR1⁺ CD8⁺ T cells.

FIG. 9 is a graph plotting % CX3CR1⁺ Granzyme CD8⁺ T cells among human peripheral blood mononuclear cells (PBMC) cultured with graded concentrations of rh-IL-15 for 24 hours in vitro. Data show the mean %±SEM. *P<0.05, **P<0.01, n=5, One-way ANOVA.

FIGS. 10A and 10B are graphs plotting tumor size in wild type (FIG. 10A) or CX3CR1 KO (FIG. 10B) mice that were inoculated with B16-OVA melanoma cells and then treated with intratumor (i.t.) injection of anti-PD-1 antibody (G4, 20 μg), soluble IL-15 (sIL-15) complex (mIL-15: 0.1 μg plus IL-15Ra chain: 0.6 μg), or both, for 3 doses on days 7, 10, and 13. Data show the mean size of tumors ±SEM (n=5).

FIG. 11 is a graph showing that IL-15 and PD-1 antibodies increased CX3CR1⁺ effector T cells within tumor tissue. B16-OVA melanomas were treated by i.t. injection of anti-PD-1 antibody (G4), soluble IL-15 (sIL-15) complex, or both, for 3 doses on days 7, 10, and 13. The % CX3CR1⁺ Granzyme B⁺ cells among CD11 CD8⁺ TILs was measured on day 10 after tumor injection, which was 3 days after one dose of the indicated reagents. *P<0.05; **P<0.01 (two-tailed, unpaired t test, n=6).

FIGS. 12A and 12B are graphs showing that IL-15 blockade decreased CX3CR1⁺ effector cells within tumor tissues. Poly IC and anti-CD40 demonstrated antitumor activity in treatment of B16-OVA tumors (FIG. 12A) and induction of CX3CR1⁺ effector CD8+ T cells within tumor tissues (FIG. 12B; TILs analyzed on day 11). Anti-IL-15 antibody (administered by peritumoral injection on days 7, 8, 9 after tumor injection) abolished the increase in CX3CR1⁺ effector CD8⁺ T cells that was induced by poly IC and anti-CD40. *P<0.05 by Mann Whitney test.

FIG. 13 is a graph plotting tumor size after treatment with IL-15, chemotherapy, or both, demonstrating that IL-15 promotes the efficacy of chemotherapy. B16F10 mouse melanoma tumors were treated with i.p. injection of carboplatin and paclitaxel (CP) on day 10 after tumor injection. Soluble IL-15 (sIL-15) complex (mIL-15: 0.1 mg plus IL-15Ra chain: 0.6 mg) was administered on days 7, 10, and 13 after tumor injection.

FIGS. 14A to 14C show that Bim up-regulation is associated with PD-1 expression in metastatic melanoma (MM) patients. FIG. 14A is a graph plotting the frequency of Bim⁺ among CD11a^{high} CD8⁺ T cells from peripheral blood of MINI patients (n=29, mean±SD) and healthy donors (HD, n=20). **P<0.01. FIG. 14B is a graph demonstrating the positive correlation of Bim and PD-1 expression in CD11a^{high}CD8⁺ T cells of MM patients (n=26). FIG. 14C is an image showing co-staining of PD-1 and Bim in melanoma tissues. The black arrow indicates a Bim and PD-1 double positive tumor infiltrating lymphocytes (TILs), while the white arrow indicates a PD-1 single positive TILs. The inset is an enlarged image (400×).

FIGS. 15A to 15D illustrate changes in Bim⁺ CD8⁺ T cells in response to PD-1 ICI therapy in patients with MM. FIG. 15A is a graph plotting percentages of changes in the frequency (%) of Bim⁺ CD8⁺ T cells in MM patients with progressive diseases (P, n=7) and responders (R, n=6) at 12-weeks after PD-1 therapy. **P<0.01, error bars, median with interquartile ranges. FIG. 15B is a series of images of metastatic melanoma (white arrows) in one patient with pseudo-progression at 12 weeks after PD-1 therapy. FIG. 15C is a graph plotting % Bim⁺ CD8⁺ T cells of the patient of FIG. 15B at baseline, 12 weeks, and 16 weeks after PD-1 therapy. FIG. 15D is a graph plotting the % change of Bim⁺ CD8⁺ T cells in a second cohort of melanoma patients (total 38) at 12 weeks after PD-1 therapy.

FIGS. 16A and 16B illustrate a model of negative correlation between changes in Bim⁺ CD8⁺ T cells and CX3CR1⁺ Granzyme CD8⁺ T cells. FIG. 16A is a graph plotting a liner relationship model, and FIG. 16B plots a curvilinear relationship model. In the model, when a decrease in Bim⁺ CD8⁺ T cells reaches a certain level, an increase of CX3CR1⁺ Granzyme B⁺ CD8⁺ T cells will take off.

FIG. 17 illustrates a gating and data collection strategy. Whole PBMC are stained with the indicated antibodies followed with gating on appropriate cell populations. Each staining and flow analysis is done in triplicate for final calculation of % Bim⁺ CD8⁺ and % CX3CR1⁺ Granzyme B⁺ CD8⁺ T cells.

FIGS. 18A and 18B are a pair of graphs illustrating potential collective thresholds of changes for the two biomarkers. The horizontal line indicates a threshold of change for Bim⁺ CD8⁺, and the vertical line indicates a threshold of change for CX3CR1⁺ Granzyme B⁺ CD8⁺ T cells in either a linear (FIG. 18A) or a curvilinear (FIG. 18B) relationship. The shaded areas indicate a range of two markers that collectively can predict a durable clinical response.

### DETAILED DESCRIPTION

This document provides materials and methods for identifying patients as being likely to respond to combined CIT, as well as materials and methods for determining optimal therapies and therapeutic timing, and methods and materials for treating cancer. For example, this document provides methods and materials for identifying a subject (e.g., a mammal such as a human) as having an increase in the percentage of CD8⁺ T cells that are CX3CR1⁺ (also referred to % CX3CR1⁺ CD8⁺ T cells), where the cells are from, e.g., a tumor or the peripheral blood, and classifying that subject as likely to be responsive to treatment with a combination of immunotherapy (e.g., ICI) and chemotherapy (known as CIT). The increase can be relative to a corresponding control percentage, or relative to a previously established percentage for the subject being assessed. In some cases, the methods also can include treating the identified subject with CIT. As described herein, an increased % CX3CR1⁺ CD8⁺ T cells can be related to increased efflux of chemotherapy drugs, as well as increased effector memory phenotype.

Having the ability to identify mammals as having a tumor that is likely to respond to a certain treatment (e.g., CIT, ICI, or a combination of CIT and ICI) can allow those mammals to be properly identified and treated in an effective and reliable manner. For example, the disease treatments described herein (e.g., CIT, ICI, and a combination of CIT and ICI) can be used to treat cancer patients identified as having a tumor that is identified as likely to respond to such treatment.

The methods provided herein, in some embodiments, can include identifying a subject as having an increased % CX3CR1⁺ Granzyme B⁺ CD8⁺ T cells, increased % CX3CR1⁺ CD8⁺ T cells in combination with decreased % Bim⁺ CD8⁺ T cells, or increased % CX3CR1⁺ Granzyme B⁺ CD8⁺ T cells in combination with decreased % Bim⁺ CD8⁺ T cells, relative to a corresponding control or previously established percentage for that subject. Subjects who are identified according to any of these criteria can be classified as being likely to respond to CIT. Conversely, subjects who are identified as not having an increased % CX3CR1⁺ CD8⁺ T cells, increased % CX3CR1⁺ Granzyme B⁺ CD8⁺ T cells, increased % CX3CR1⁺ CD8⁺ T cells in combination with decreased % Bim⁺ CD8⁺ T cells, or increased % CX3CR1⁺ Granzyme B⁺ CD8⁺ T cells in combination with decreased % Bim⁺ CD8⁺ T cells, relative to a corresponding control or previously established percentage for that subject, can be classified as not being as likely to respond to CIT.

The term “increased” as used herein with respect to % CX3CR1⁺ CD8⁺ T cells or % CX3CR1⁺ Granzyme B⁺ CD8⁺ T cells refers to a percentage that is greater (e.g., at least 5% greater, at least 10% greater, at least 25% greater, at least 50% greater, 5 to 10% greater, 10 to 25% greater, 25 to 50% greater, 50 to 75% greater, at least 2-fold greater, at least 3-fold greater, at least 5-fold greater, 2- to 3-fold greater, or 3- to 5-fold greater) than a reference % CX3CR1⁺ CD8⁺ T cells or % CX3CR1⁺ Granzyme B⁺ CD8⁺ T cells. The term “decreased” as used herein with respect to % Bim⁺ CD8⁺ T cells refers to a percentage that is less (e.g., at least 5% less, at least 10% less, at least 25% less, at least 50% less, at least 75% less, at least 90% less, at least 95% less, 5 to 10% less, 10 to 25% less, 25 to 50% less, 50 to 75% less, or 75 to 100% less) than a reference % Bim⁺ CD8⁺ T cells.

The terms “reference %,” “reference percentage” and “reference level” (also referred to herein as “corresponding control %,” “corresponding control percentage,” and “corresponding control level”), as used herein with respect to CX3CR1⁺ CD8⁺ T cells, CX3CR1⁺ Granzyme B⁺ CD8⁺ T cells, and Bim⁺ CD8⁺ T cells, refer to the % CX3CR1⁺ cells, % CX3CR1⁺ Granzyme B cells, or % Bim+ cells in a sample of CD8⁺ T cells taken from a subject at baseline (e.g., prior to treatment with ICI or chemotherapy).

The presence of an increased % CX3CR1⁺ CD8⁺ T cells, increased % CX3CR1⁺ Granzyme B⁺ CD8⁺ T cells, or decreased % Bim⁺ CD8⁺ T cells can be determined using, for example, flow cytometry according to the methods described in the Examples herein. In some cases, methods such as time of flight mass cytometry (cyToF), single cell or sorted cell-RNA-sequencing analysis cell staining, western blotting, multiplex immunofluorescence imaging analysis, immunohistochemistry (IHC), or other immunological techniques can be used.

The populations of CD8⁺ T cells used in the methods provided herein can be from any suitable source within the subject. In some cases, for example, the CD8⁺ T cells are obtained from the peripheral blood of the subject, while in other cases, the CD8⁺ T cells are from a tumor within the subject. Other suitable sources include, for example, ascite samples and lymphoid organ samples.

Thus, in some embodiments, this document provides methods that include measuring the % CX3CR1⁺ cells within a population of CD8⁺ T cells obtained from a subject that has a tumor, and identifying the subject as being likely to respond to CIT when the % CX3CR1⁺ cells within the population is increased relative to a corresponding control % CX3CR1⁺ cells. The methods also can include measuring the % CX3CR1⁺ Granzyme B⁺ cells within the population of CD8⁺ T cells from the subject; in such embodiments, the subject can be identified as likely to respond to CIT when the % CX3CR1⁺ Granzyme B⁺ cells within the population is increased relative to a corresponding control percentage. In some cases, the methods also may include administering the CIT to the subject.

This document also provides methods that can include measuring the % CX3CR1⁺ cells in a first population of CD8⁺ T cells obtained from a subject with a tumor prior to CIT, measuring the % CX3CR1⁺ cells in a second population of CD8⁺ T cells obtained from the subject after CIT, and identifying the subject as being responsive to the CIT when the % CX3CR1⁺ cells in the second population is greater than the % CX3CR1⁺ cells in the first population. In some cases, the methods can include measuring the % CX3CR1⁺ Granzyme B⁺ cells in the first and second populations of CD8⁺ T cells, and identifying the subject as being responsive to the CIT when the % CX3CR1⁺ Granzyme B⁺ cells in the second population is greater than the % CX3CR1⁺ Granzyme B⁺ cells in the first population. In some cases, the methods also can include administering the CIT to the subject.

As described herein, the percentage of Bim⁺ cells in a population of CD8⁺ T cells can be inversely correlated with the percentage of CX3CR1⁺ or CX3CR1⁺ Granzyme B⁺ cells in the population. In some cases, therefore, the methods provided herein also can utilize the % Bim⁺ CD8⁺ T cells as an indicator that a subject is likely to respond to CIT or another therapy. Such methods can include, for example, measuring the % Bim⁺ CD8⁺ T cells within a population of CD8⁺ T cells evaluated for CX3CR1, or CX3CR1 and Granzyme B, and identifying the subject as being likely to respond to CIT when the % Bim⁺ CD8⁺ T cells within the population is decreased relative to a corresponding control % Bim⁺ CD8⁺ T cells.

In some cases, the change in % CX3CR1⁺ CD8⁺ T cells (or % CX3CR1⁺ Granzyme B⁺ T cells) and the change in % Bim⁺ CD8⁺ T cells from a reference percentage in a sample from a subject (e.g., before treatment of the subject with ICI, CIT, or chemotherapy) can be used to determine a therapy that is likely to benefit the subject. Samples containing CD8⁺ T cells obtained from the subject before and after treatment (e.g., with an ICI therapy such as anti-PD-1 therapy) can be assessed to determine the % CX3CR1⁺ and % Bim⁺ CD8⁺ T cells in the samples, and a further treatment regimen can be determined based, at least in part, on whether the changes in % CX3CR1⁺ CD8⁺ T cells and Bim⁺ CD8⁺ T cells reach certain predetermined thresholds.

For example, when the % CX3CR1⁺ cells in the second population is increased by at least a predetermined threshold relative to the % CX3CR1⁺ cells within the first population, and the % Bim⁺ cells in the second population is decreased by at least a predetermined threshold relative to the % Bim⁺ cells in the first population, it may be determined that they subject is likely to benefit from a therapy that can increase tumor immunogenicity (e.g., radiation therapy). When the % CX3CR1⁺ cells in the second population is increased by less than the predetermined CX3CR1⁺ threshold and the % Bim⁺ cells in the second population is decreased by at least the predetermined Bim⁺ threshold, it may be determined that the subject is likely to benefit from cytokine therapy (e.g., treatment with IL-15) combined with PD-1 blockade therapy. When the % CX3CR1⁺ cells in the second population is increased by at least the predetermined CX3CR1⁺ threshold and the % Bim⁺ cells in the second population is increased, unchanged, or decreased by less than the predetermined Bim⁺ threshold, it may be determined that the subject is likely to benefit from CIT. When the % CX3CR1⁺ cells in the second population is increased by less than the predetermined CX3CR1⁺ threshold and the % Bim⁺ cells in the second population is increased, unchanged, or decreased by less than the predetermined Bim⁺ threshold, it may be determined that the subject is likely to benefit from an ICI therapy other than PD-1 blockade therapy (e.g., anti-TIGIT (T cell immunoreceptor with Ig and ITIM domains) therapy and/or anti-Tim 3 therapy), optionally in combination with chemotherapy.

The predetermined thresholds can be established using methods such as those described in the examples herein. In some embodiments, a predetermined CX3CR1 threshold can be an increase of at least 25% (e.g., at least 30%, at least 35%, at least 40%, at least 50%, at least 55%, at least 60%, at least 65%, at least 70%, at least 75%, at least 80%, at least 85%, at least 90%, or at least 95%), and a predetermined Bim threshold can be a decrease of at least 5% (e.g., at least 10%, at least 15%, at least 20%, at least 25%, at least 30%, at least 35%, at least 40%, at least 45%, or at least 50%).

The populations of CD8 T cells used in the methods described herein can be obtained from a subject at any suitable time. For example, CD8⁺ T cells can be obtained before or after (e.g., six, 12, 16, 32, two to four, four to six, six to eight, eight to 12, 12 to 16, 16 to 32, or more than 32 weeks after) treatment with CIT, before or after treatment with chemotherapy (e.g., paclitaxel and/or carboplatin), or before or after ICI therapy (e.g., with an anti-PD-1 or anti-PD-L1 antibody), or when disease progresses.

The subject can be a mammal (e.g., a human, non-human primate, mouse, rat, rabbit, pig, sheep, dog, cat, or horse), and can have a tumor such as, without limitation, a melanoma (e.g., a metastatic melanoma), a gastrointestinal tumor, a genitourinary tumor, a non-small cell lung cancer, or a breast tumor.

In addition, this document provides methods that can be used to expand CX3CR1⁺ CD8 T cells, either in vitro, ex vivo, or in vivo. Such methods can utilize interleukin-15 (IL-15) to stimulate expansion of the cells, as described in Example 8 herein; methods also can utilize IL-12, IL-2 and IL-7, and/or fractalkine (a CX3CR1 ligand) to stimulate expansion of the cells. Thus, in some embodiments, the methods provided herein can include obtaining a population of CX3CR1⁺ CD8⁺ T cells from a subject and then contacting the population with IL-15 in order to expand the population. In some cases, the methods can further include returning at least a portion of the expanded population to the subject from which they were obtained (e.g., to combat a tumor, for example). Methods for in vivo use can include, for example, measuring the % CX3CR1⁺ cells in a first population of CD8⁺ T cells obtained from a subject with a tumor, administering IL-15 to the subject, measuring the % CX3CR1⁺ cells in a second population of CD8⁺ T cells obtained from the subject after IL-15 administration to demonstrate that the % CX3CR1⁺ cells within the second population has increased relative to the % CX3CR1⁺ cells in the first population.

In some embodiments, once a subject has been identified as having an increased % CX3CR1⁺ CD8⁺ T cells, increased % CX3CR1⁺ Granzyme B⁺ CD8⁺ T cells, or increased % CX3CR1⁺ or % CX3CR1⁺ Granzyme B⁺ CD8⁺ T cells in combination with decreased % Bim⁺ CD8⁺ T cells, the subject can be treated with one or more cancer therapies. Examples of such therapies include, without limitation, chemotherapies such as paclitaxel, carboplatin, cisplatin, doxorubicin, or gemcitabine, ICI therapies targeted to PD-1 or PD-L1, a combination of ICI therapy and chemotherapy (CIT), and radiation. Methods for administering such therapies are known in the art. Administration can be, for example, parenteral (e.g., by subcutaneous, intrathecal, intraventricular, intramuscular, or intraperitoneal injection, or by intravenous drip). Administration can be rapid (e.g., by injection) or can occur over a period of time (e.g., by slow infusion or administration of slow release formulations). In some embodiments, administration can be topical (e.g., transdermal, sublingual, ophthalmic, or intranasal), pulmonary (e.g., by inhalation or insufflation of powders or aerosols), or oral. In addition, a therapy can be administered prior to, after, or in lieu of surgical resection of a tumor.

A cancer therapy (e.g., chemotherapy or immunotherapy, or a CIT) can be administered to a mammal in an appropriate amount, at an appropriate frequency, and for an appropriate duration effective to achieve a desired outcome (e.g., to increase progression-free survival, reduce tumor size, etc.). In some cases, a therapy can be administered to a subject having cancer to reduce the progression rate of the cancer by at least 5 percent (e.g., at least 5 percent, at least 10 percent, at least 25 percent, at least 50 percent, at least 75 percent, or 100 percent). For example, the progression rate can be reduced such that no additional cancer progression is detected. Any appropriate method can be used to determine whether or not the progression rate of cancer is reduced. For skin cancer (e.g., melanoma), for example, the progression rate can be assessed by imaging tissue at different time points and determining the amount of cancer cells present. The amounts of cancer cells measured in tissue at different times can be compared to determine the progression rate. After treatment, the progression rate can be determined again over another time interval. In some cases, the stage of cancer after treatment can be determined and compared to the stage before treatment to determine whether or not the progression rate has been reduced.

In some cases, a therapy can be administered to a subject having cancer under conditions where progression-free survival is increased (e.g., by at least 5, at least 10, at least 25, at least 50, at least 75, or at least 100 percent) as compared to the median progression-free survival of corresponding subjects having untreated cancer, or the median progression-free survival of corresponding subjects having cancer and treated with other therapies. Progression-free survival can be measured over any length of time (e.g., one month, two months, three months, four months, five months, six months, or longer).

An effective amount of a composition containing a molecule as provided herein can be any amount that reduces tumor size, reduces the progression rate of cancer, increases the progression-free survival rate, or increases the median time to progression without producing significant toxicity to the mammal. Optimum dosages can vary depending on the relative potency of individual therapies (e.g., antibodies and chemotherapeutics), and can generally be estimated based on EC₅₀ found to be effective in in vitro and in vivo animal models. Typically, dosage is from 0.01 μg to 100 g per kg of body weight. For example, an effective amount of an antibody or fusion protein can be from about 1 mg/kg to about 100 mg/kg (e.g., about 5 mg/kg, about 10 mg/kg, about 20 mg/kg, about 50 mg/kg, about 75 mg/kg, about 5 to 10 mg/kg, about 10 to 20 mg/kg, about 20 to 50 mg/kg, or about 75 to 100 mg/kg). If a particular subject fails to respond to a particular amount, then the amount of the therapy can be increased by, for example, two-fold. After receiving this higher concentration, the subject can be monitored for both responsiveness to the treatment and toxicity symptoms, and adjustments made accordingly. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the cancer may require an increase or decrease in the actual effective amount administered.

The frequency of administration can be any frequency that reduces tumor size, reduces the progression rate of cancer, increases the progression-free survival rate, or increases the median time to progression without producing significant toxicity to the subject. For example, the frequency of administration can be once or more daily, biweekly, weekly, monthly, or even less. The frequency of administration can remain constant or can be variable during the duration of treatment. A course of treatment can include rest periods. For example, a composition containing an immunotherapy can be administered over a two week period followed by a two week rest period, and then repeated or followed by treatment with chemotherapy. As with the effective amount, various factors can influence the actual frequency of administration used for a particular application. For example, the effective amount, duration of treatment, use of multiple treatment agents, route of administration, and severity of the cancer may require an increase or decrease in administration frequency.

An effective duration for administering a therapy can be any duration that reduces tumor size, reduces the progression rate of cancer, increases the progression-free survival rate, or increases the median time to progression without producing significant toxicity to the subject. Thus, the effective duration can vary from several days to several weeks, months, or years. In general, the effective duration for the treatment of cancer can range in duration from several weeks to several months. In some cases, an effective duration can be for as long as an individual subject is alive.

Multiple factors can influence the actual effective duration used for a particular treatment. For example, an effective duration can vary with the frequency of administration, effective amount, use of multiple treatment agents, route of administration, and severity of the cancer.

After administering a therapy to a subject with cancer, the subject can be monitored to determine whether or not the cancer was treated. For example, a subject can be assessed after treatment to determine whether or not the progression rate of the cancer has been reduced (e.g., stopped), or whether the tumor size has decreased. Any method, including those that are standard in the art, can be used to assess progression and survival rates.

The invention will be further described in the following examples, which do not limit the scope of the invention described in the claims.

### EXAMPLES

### Example 1—Materials and Methods

Patient information: The studies described herein were conducted according to Declaration of Helsinki principles. Peripheral blood and tissue samples for this study were collected after written consents were obtained. Clinical course, treatment information and outcomes in patients with metastatic melanoma who did not respond to anti-PD-1 (programmed cell death protein 1) single agent therapy were retrospectively collected. Patients who failed initial PD-1 therapy were subsequently treated with salvage paclitaxel and carboplatin combination in addition to PD-1 blockade, regardless of BRAF mutant status. Response to treatment was evaluated according to standard clinical practice guidelines using Response Evaluation Criteria In Solid Tumors (RECIST) criteria.

Flow analysis of human T cells isolated from peripheral blood: PBMC samples were collected from healthy donors or patients with melanoma. Antibodies for CD45, CD3, CD8, CX3CR1 (2A9-1), CD11a (HI111) and PD-1 (EH12.2H7) were purchased from BioLegend (San Diego, Calif.); anti-human Granzyme B (GB11) was purchased from Life Technologies (Waltham, Mass.). CD8⁺ T cells were first stained for surface markers (CX3CR1, etc.), followed by intracellular staining for Granzyme B. To initiate CTL (cytotoxic T lymphocyte) function, cells were briefly stimulated with phosphomolybdic acid (PMA) and ionomycin (Sigma; St. Louis, Mo.) for 5 hours in the presence of anti-CD107a antibody (H4A3), followed by intracellular staining of anti-IFN-γ antibody (4S.B3). Flow cytometry analysis was performed using FlowJo software (Tree Star; Ashland, Oreg.).

RNA-seq and bioinformatics analysis: Total RNA was extracted from flow sorted cells using an RNeasy Mini kit (Qiagen; Hilden, Germany) and checked for quality by Bioanalyzer (RNA 6000 Pico kit; Agilent; Santa Clara, Calif.). A total of 1 ng of RNA was used to generate double stranded cDNA using SMARTER™ Ultra Low RNA kit for Illumina (Takara; Mountain View, Calif.). Full length, double stranded cDNA was quantified and subjected to RNA-Seq library construction. A total of 250 pg of cDNAs were used to construct indexed libraries using NEXTERA® XT DNA Sample Preparation kit (Illumina; San Diego, Calif.). The cDNA and NGS libraries were quantified using Bioanalyzer (High Sensitivity DNA analysis kit; Agilent) and Qubit (dsDNA BR Assay kits; Life Technologies). The libraries were sequenced using the 101 bases paired-end protocol on Illumina HiSeq 2000. FASTQ formatted raw files from each sample were mapped and aligned to reference hg19.

The MAPRSeq workflow for mRNA was used to align raw FASTQ reads, using TopHat2 to the relevant genome. The BAM files thus obtained were passed through other tools for further analysis. Fusion detection was done using a module from the TopHat aligner, called TopHat-Fusion. Raw and normalized gene and exon counts were generated by FeatureCounts, which uses the ENSEMBL GRCh38.78 gene definitions. An in-house tool (RVBoost; Wang et al., Bioinformatics, 2014, 30(23):3414-3416), which uses UnifiedGenotyper from GATK, was employed to report single nucleotide variants present in the data. Finally, the RSeQC module created a variety of QC plots and graphs to ensure that the quality of samples was good and reliable for use in further downstream analyses (e.g., differential expression and pathway analysis). The R-based tool from Bioconductor, edgeR v3.8.6, was used to perform the differential expression analysis comparing the various sample groups. Genes encoded by mRNA that had an absolute log 2 fold change >1.5 were considered to be significantly differently expressed. Heatmaps were created using the heatmap.2 function of the gplots package from R.

Immunochemistry staining of melanoma tissues: Paraffin-embedded tissue sections were cut into 5 m sections, deparaffinized in xylene, and rehydrated in a graded series of alcohols. Antigen retrieval was performed by heating tissue sections in Target Retrieval Solution pH 6.0 (Dako #S1699) at 98° C. for 30 minutes. Sections were cooled on the bench for 20 minutes, washed in running DH20 for 5 minutes, and then incubated for 5 minutes in wash buffer. Sections were then blocked for 5 minutes with Endogenous Peroxidase Block (Dako #S2001), washed, and blocked for 5 minutes in Protein Block Serum Free (Biocare Medical #X0909). Slides were incubated for one hour in mouse monoclonal anti-human Granzyme B (Dako #M7235) diluted 1:50 in Antibody Diluent with Background Reducers (Dako #3022). Sections were washed and incubated 15 minutes each in mouse probe and mouse polymer AP (Mach 3 Mouse AP Polymer Detection Kit, Biocare Medical #M3M532L). Sections were incubated for 5 minutes in Warp Red Chromogen (Biocare Medical #WR806H) for visualization. Subsequently, sections were incubated for 5 minutes in 80° C. Citrate Buffer pH 6, rinsed in wash buffer and incubated in Protein Block Serum Free for 5 minutes. Rabbit anti-human CX3CR1 (Invitrogen PA5-32713) was applied to sections at 1:500 dilution and incubated for one hour at room temperature. Sections were washed and incubated for 15 minutes each in rabbit probe and rabbit polymer HRP (Mach 3 Rabbit HRP Polymer Detection kit, Biocare Medical # M3R531L) and visualized for one minute in DAB (Biocare Medical #BDB2004L). Sections were counterstained and coverglass mounted with PERMOUNT™.

Stimulation and culture of human T cells: Human CD8⁺ T cells were purified using a human CD8⁺ T cell enrichment kit (Stemcell). CD8⁺ T cells were incubated with chemotherapy drugs (paclitaxel, carboplatin, or doxorubicin), either alone or with T cell activators (DYNABEADS®, human T-activator CD3/CD28 beads) for 24-48 hours, followed with staining for CX3CR1 and Granzyme B. ABCB1 inhibitor PGP4008 was purchased from Enzo Life Sciences (Farmingdale, N.Y.).

Drug efflux assay in T cells: Human primary CD8⁺ T cells were isolated from peripheral blood and incubated (loading) with Rh123 (10 μg/ml) on ice for 30 minutes, or with doxorubicin (Dox, 1 μg/ml) at 37° C. for 60 minutes in water bath. After the loading process, cells were washed and cultured at 37° C. for 60 minutes (efflux), stained for cell surface markers, and analyzed by flow cytometry. The ABCB1 inhibitor PGP-4008 was added at 1-5 μM during the efflux process.

Animal models for chemo-immunotherapy: Both wild type and CX3CR1-deficient (KO) mice in the C57BL/6 background were purchased from Jackson Lab (Bar Harbor, Me.) and maintained under pathogen-free conditions. B16F10 mouse melanoma cells (1×10⁵) were subcutaneously (s.c.) injected into mice in the right flank, followed by i.p. injection of 100 g anti-PD-1 (G4), anti-PD-L1 (10B5), or control IgG starting on day 7, for a total of five doses at 3-day intervals. Carboplatin (40 μg/g plus paclitaxel (10 μg/g body weight) were injected i.p. once, either on day 7 or on day 10 after tumor injection. CTL function of tumor-infiltrating CD8⁺ T cells was measured by briefly stimulating them with PMA and ionomycin (Sigma) for 5 hours in the presence of anti-CD107a antibody (1D4B), followed by intracellular staining with anti-IFN-γ antibody (XMG1.2). Perpendicular tumor diameters were measured using a digital caliper and tumor sizes were calculated as length×width. Tumor growth was evaluated every 2 to 3 days until ethical endpoints, when all mice were euthanized.

T cell transfer therapy: Spleen cells isolated from OT-1 mice expressing OVA-antigen-specific TCR were cultured with OVA peptide (1 μg/ml) and rhIL-2 (10 IU/ml) for 48 hours. CX3CR1⁺ and CX3CR1⁻ CD8⁺ T cells were sorted after culture on the day of T cell transfer. Once B16-OVA mouse melanoma tumors were established, around day 7 after tumor cell injection (5×10⁵ cells per mouse, s.c.), the animals were treated by i.t. injection of CX3CR1⁺ or CX3CR1⁻ CD8⁺ T cells at equal numbers (2 to 3×10⁵ T cells per mouse) for a total of three doses on days 7, 10, and 13 after tumor injection.

Statistics: The Mann-Whitney test was used to compare independent groups (function or subsets of CD8⁺ T cells). The impact of chemotherapy and anti-PD antibody on tumor growth were analyzed by two-way ANOVA. Comparisons of the impact of ABCB1 inhibitors on the efflux of drug were analyzed with one-way ANOVA due to the numerical independent variables. The survival of animals was analyzed by Log-rank Mantel-Cox test. All statistical analyses were performed using GraphPad Prism software 5.0 (GraphPad Software, Inc.; San Diego, Calif.). A P value <0.05 was considered statistically significant.

### Example 2—Patients Who Failed PD-1 Blockade Benefit from CIT

A large fraction of cancer patients (60-70%) who receive PD-1 blockade alone are resistant to PD-1 therapy or experience subsequent disease progression (Robert et al., N Engl J Med, 2015, 372(26):2521-2532; Robert et al., N Engl J Med, 2015, 372(4):320-330; and Ribas et al., JAMA, 2016, 315(15):1600-1609). Some of these patients, however, benefited from late-line or salvage treatment with conventional chemotherapy. Since the safety and efficacy profile of CIT have been demonstrated in NSCLC patients (Rizvi et al., J Clin Oncol, 2016, 34(25):2969-2979; and Langer et al., Lancet Oncol, 2016, 17(11):1497-1508), a number of patients who had evidence of disease progression with initial PD-1 blockade monotherapy were empirically treated with chemotherapy in addition to continued anti-PD-1 antibody (Yan et al., “The Mayo Clinic experience in patients with metastatic melanoma who have failed previous pembrolizumab treatment,” ASCO Meeting Abstracts. 2016, 34((15_suppl)):e21014). To minimize toxicity, a short-term chemotherapy (2-6 cycles) was combined with anti-PD-1 therapy that was maintained thereafter. Among 19 patients who did not respond to anti-PD-1 (Pembrolizumab) antibody and received chemotherapy (carboplatin, paclitaxel, temozoromed, or dacarbazine,), a complete follow up identified 5 patients demonstrating disease control, with an objective response rate (ORR) of 26.3% according to the RECIST criteria (Seymour et al., Lancet Oncol, 2017, 18(3):e143-e152).

### Example 3—CX3CR1 Identifies T Cells that Respond to PD-1 Monotherapy and CIT

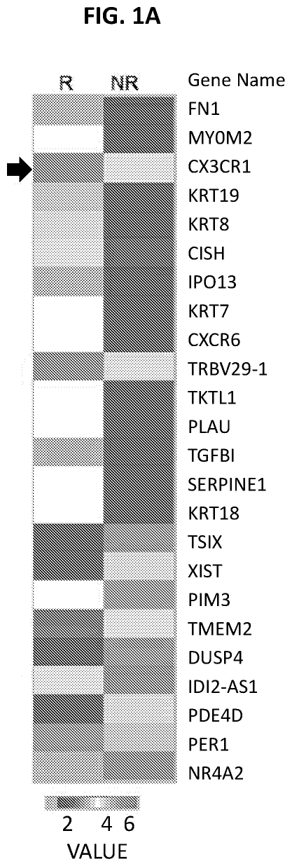

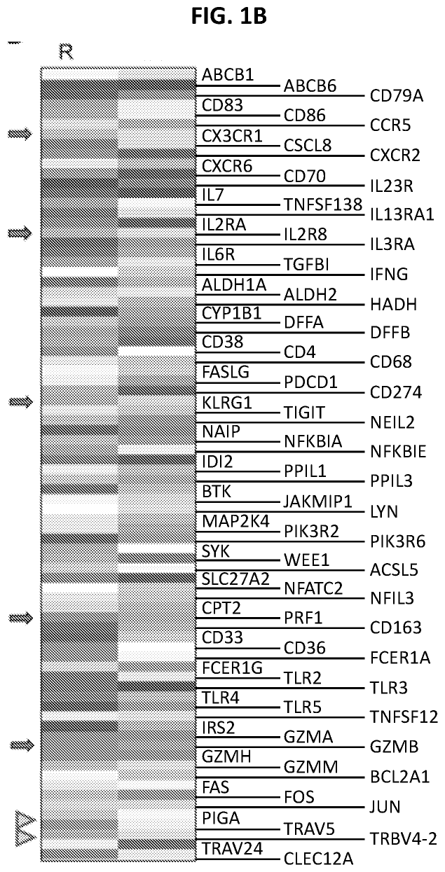

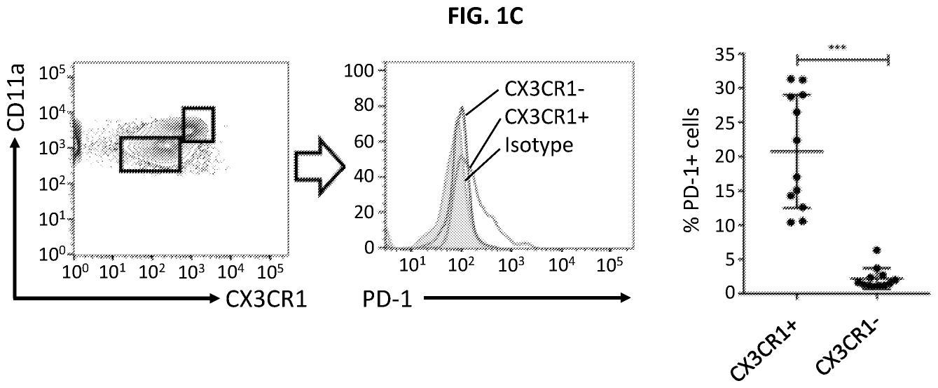

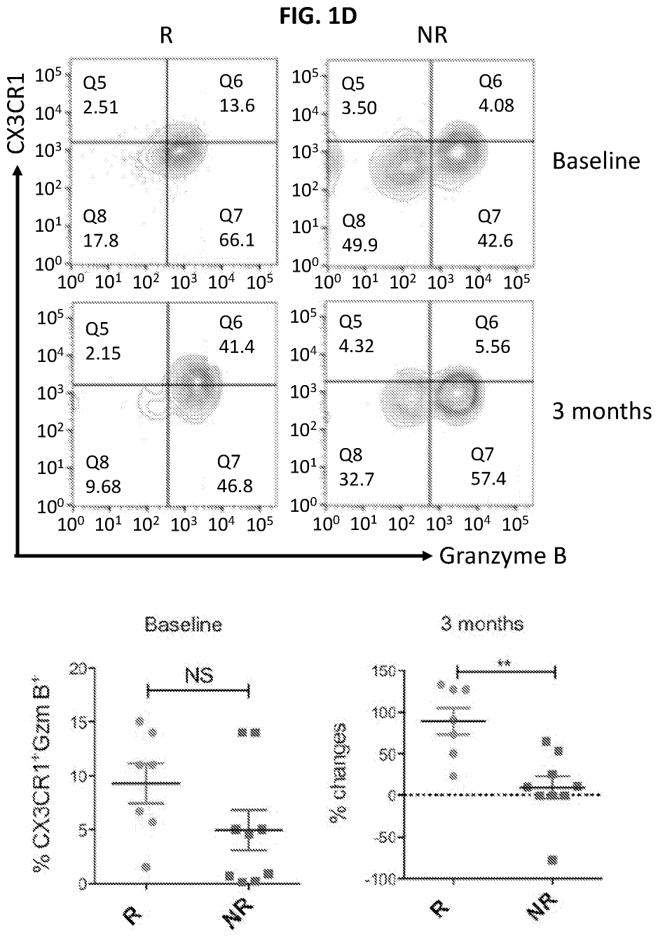

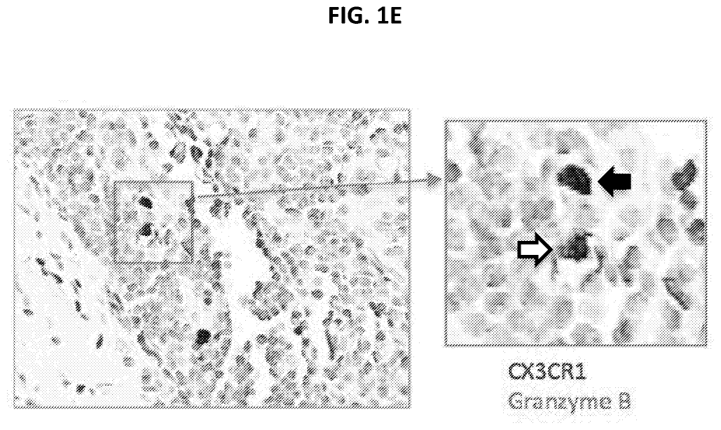

Studies were conducted to seek biomarkers for identifying responders to anti-PD-1 therapy, in order to predict and increase the efficacy of chemo-immunotherapy. First, subsets of tumor-reactive CD8⁺ T cells were examined in the peripheral blood of cancer patients to identify those that would be responsive to anti-PD-1 monotherapy. Further studies were directed at determining whether the responsive T cell population would be preserved during chemotherapy and would still be responsive to anti-PD-1 therapy. To that end, RNA-seq analysis was performed with of tumor-reactive CD11a^{high}PD-1⁺ CD8⁺ T cells (Liu et al., Oncoimmunology, 2013, 2(6):e23972), and gene transcription was compared between responders and non-responders at baseline prior to PD-1 therapy. Among the top genes with increased expression (ratio >1.5) in responders compared to non-responders, transcription of CX3CR1 was increased in the tumor-reactive CD8⁺ T cells in the peripheral blood of responders to PD-1 therapy (FIG. 1A). Of note, there was over-representation of TCRβ V29-1 among CD11a^{high} PD-1⁺CD8⁺ T cells in responders prior to PD-1 therapy, suggesting there might be a monoclonal expansion of tumor-reactive T cells that would eventually be responsive to anti-PD-1 therapy.

The gene expression in CD11a^{high} CD8⁺ T cells isolated and sorted from the peripheral blood of 3 months after anti-PD-1 treatment was then compared between responders and non-responders. As shown in FIG. 1B, the responders harbored more effector memory CD8⁺ T cells than non-responders based on their higher (>2-fold change) expression of CX3CR1, CD122 (IL-2 receptor beta chain), KLRG1 (effector differentiation marker), perforin, and Granzyme B (effector molecules). However, IFN-γ expression was unexpectedly increased in CD8⁺ T cells of non-responders compared to responders. Despite its role in antitumor activity, IFN-γ plays a role in inducing apoptosis of effector cells and limiting memory cell generation (Liu and Janeway, J Exp Med, 1990, 172(6):1735-1739; Prabhu et al., J Virol, 2013, 87(23):12510-12522; and Refaeli et al., J Exp Med, 2002, 196(7):999-1005). These results therefore suggested further scrutiny of the role of IFN-γ expressed by tumor-reactive T cells in response to anti-PD-1 therapy. Although RNA-seq analysis was performed on a different cohort of patients (FIG. 1B), the increase in CX3CR1 expression was consistent with the observation at baseline (FIG. 1A). Interestingly, over-representation of TCRVα5 and TCRVβ4-2 also was observed among CD11a^{high} CD8⁺ T cells in responders after PD-1 therapy, suggesting that anti-PD-1 therapy promoted an oligoclonal expansion of tumor-reactive T cells that may contribute to tumor rejection.

To further confirm whether CX3CR1 can identify PD-1 therapy-responsive CD8⁺ T cells, the expression of PD-1 was measured and compared among CX3CR1⁺ or CX3CR1⁻ CD8⁺ T cells. As shown in FIG. 1C, PD-1 was more highly expressed in CX3CR1⁺ CD8⁺ T cells than CX3CR1-CD8⁺ T cells. Since CX3CR1 and Granzyme B can be used to identify human effector memory CD8⁺ T cells in viral infection (Bottcher et al., Nat Commun, 2015, 6:8306), the ability of CX3CR1⁺ Granzyme B⁺ to identify a subset of tumor-reactive CD8⁺ T cells in the peripheral blood of cancer patients in response to anti-PD-1 immunotherapy was evaluated. Although the frequency of CX3CR1⁺ Granzyme B⁺ cells was not significantly higher in responders than in non-responders at baseline (prior to PD-1 therapy), the percentages of CX3CR1⁺ Granzyme B⁺ cells was increased in responders as compared to non-responders after anti-PD-1 treatment (FIG. 1D). In metastatic melanoma tissues (prior to PD-1 therapy), CX3CR1⁺ Granzyme B⁺ (double positive) cells also were identified as infiltrating tumor tissues (FIG. 1E). Interestingly, CX3CR1⁺ Granzyme B⁺ (double positive) cells appeared in a blood vessel within the tumor tissue, suggesting potential extravasation of CX3CR1⁺ Granzyme B⁺ cells into tumor sites from the systemic circulation. These results suggested that the CX3CR1⁺ Granzyme B⁺ phenotype identifies a subset of CD8⁺ tumor-reactive T cells that is responsive to anti-PD-1 therapy and has the potential to migrate to tumor tissues.

### Example 4—CX3CR1⁺ Granzyme B⁺ CD8⁺ T Cells Increased in the Peripheral Blood of Responders after CIT

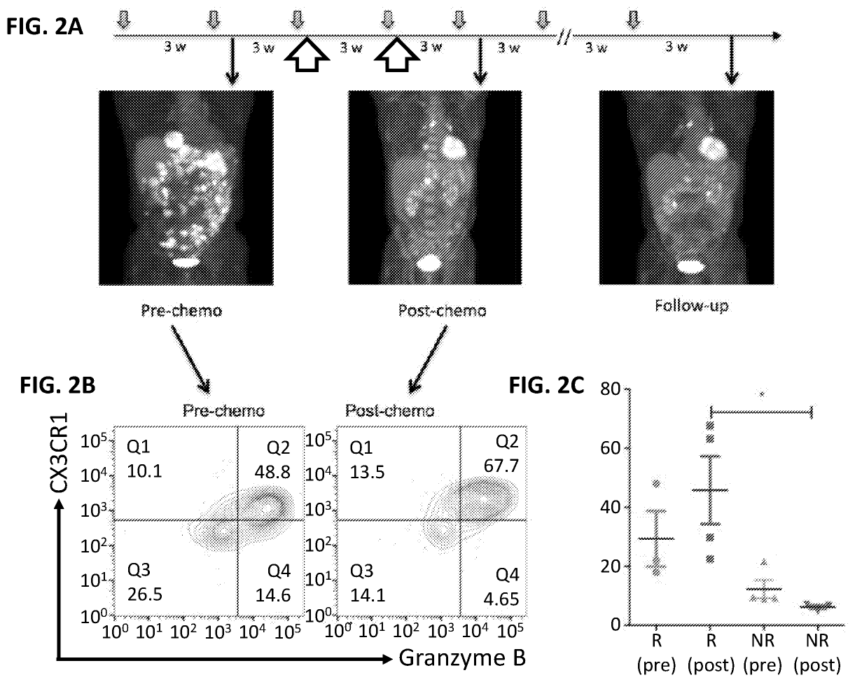

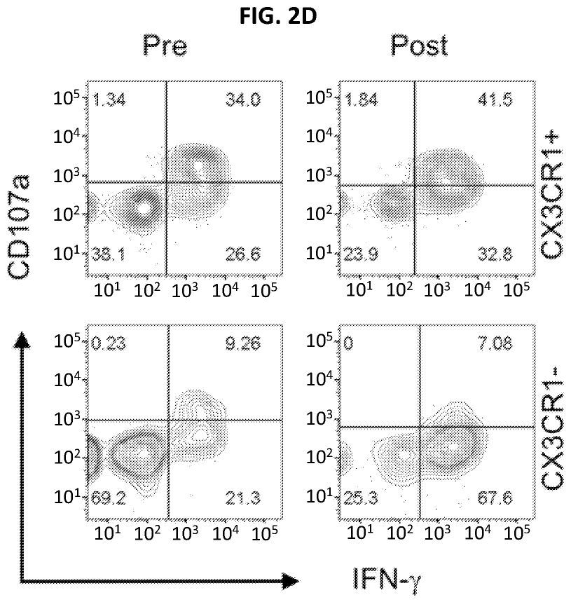

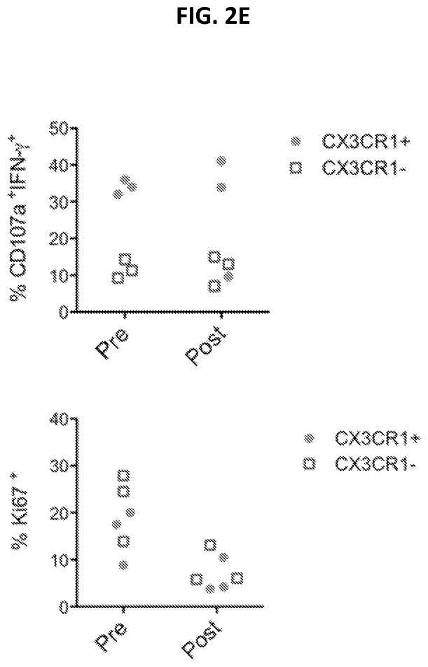

The frequency of CX3CR1⁺ Granzyme B⁺ CD8⁺ tumor-reactive T cells was examined before and after chemotherapy combined with anti-PD-1 therapy in patients with metastatic melanoma. As shown in FIG. 2A, one patient had rapid progression of metastatic melanoma in the peritoneum and liver while on treatment with anti-PD-1 antibody (pembrolizumab) alone. Treatment with carboplatin and paclitaxel (3 weeks/cycle) were therefore initiated in this patient, with continued pembrolizumab. Three weeks after the combination therapy, the patient demonstrated significant improvement of disease in the abdomen, with a dramatically reduced tumor burden. Importantly, combined CIT was stopped after two cycles, and this patient experienced an ongoing clinical benefit with maintenance single agent anti-PD-1 immunotherapy. To test whether CX3CR1⁺ Granzyme B⁺ CD8⁺ cells were preserved during chemotherapy and were still responsive to anti-PD-1 therapy, the frequency of CX3CR1⁺ Granzyme B⁺ cells in tumor-reactive (CD11a^{high} PD-1⁺) CD8⁺ T cells isolated from the patient's peripheral blood before and after the addition of chemotherapy (time points shown in FIG. 2B) was measured. One week after the addition of chemotherapy, the frequency of CX3CR1⁺ Granzyme B⁺ cells increased among CD11a^{high} PD-1⁺ CD8⁺ T cells in patients who responded to CIT, as compared to non-responders (FIG. 2B). The frequency of CX3CR1⁺ Granzyme B⁺ cells also was measured and compared in other responders and non-responders prior to and after chemotherapy. Similar to the results plotted in FIG. 2B, the frequency of CX3CR1⁺ Granzyme B⁺ cells as increased in responders as compared to non-responders post chemotherapy during CIT (FIG. 2C). Interestingly, the responders also harbored a higher frequency of CX3CR1⁺ Granzyme B⁺ cells prior to chemotherapy than non-responders, although this increase did not reach statistical significance (FIG. 2C). Further studies compared the proliferation and CTL function of CX3CR1-expressing or non-expressing CD8⁺ T cells collected from responders before and after combined therapy. As shown in FIGS. 2D and 2E, CX3CR1⁺ CD8⁺ T cells preserved their increased CTL function compared to CX3CR1⁻ CD8⁺ T cells. The levels of proliferation were comparable in CX3CR1⁺ and CX3CR1⁻ CD8⁺ T cells before chemotherapy, while their proliferation tended to decrease in both populations of CD8⁺ T cells after chemotherapy. These results suggested that CX3CR1⁺ CD8⁺ T cells can withstand chemotherapy, and that pre-existing of CX3CR1⁺ Granzyme B⁺ tumor-reactive CD8⁺ T cells may be required for clinical responses to CIT.

### Example 5—the Drug Efflux Ability of CX3CR1⁺ CD8⁺ T Cells

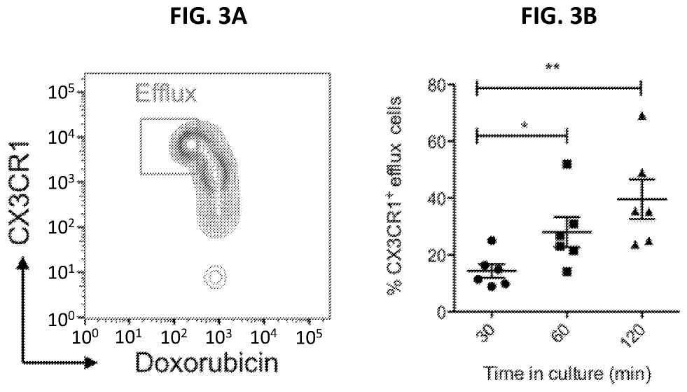

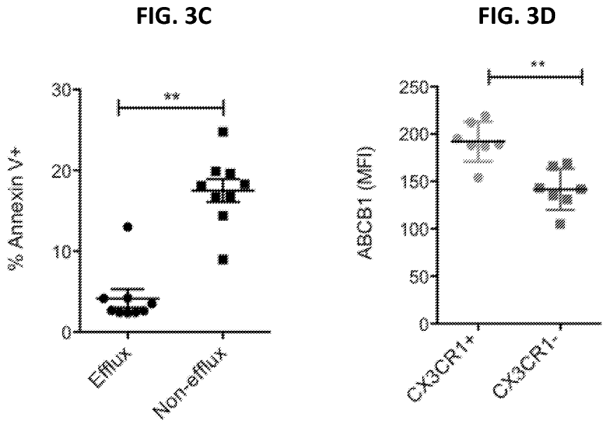

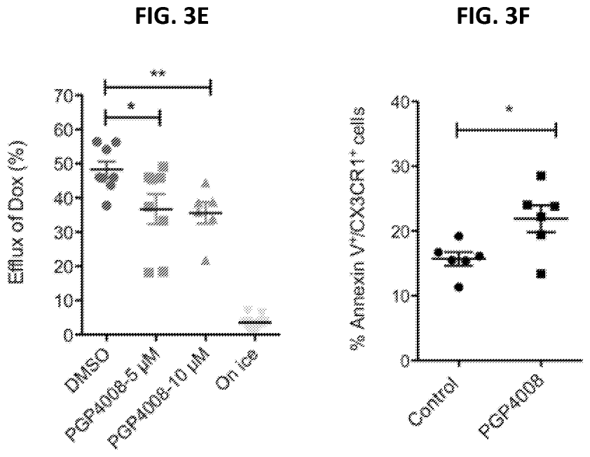

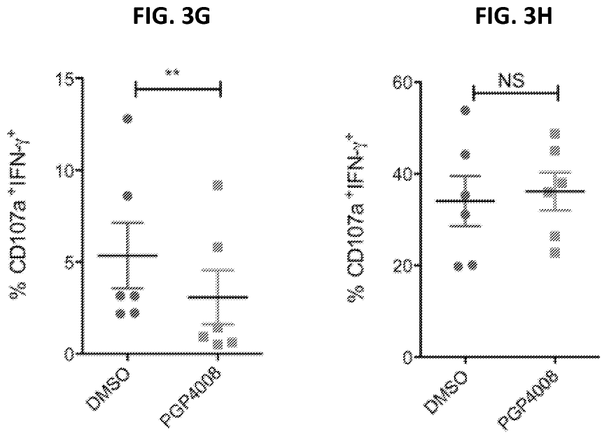

The mechanisms by which CX3CR1⁺ CD8⁺ T cells withstand chemotherapy were examined. High multidrug efflux capacity can confer upon CD8⁺ T cells the ability to survive cytotoxic chemotherapy (Turtle et al., Immunity, 2009, 31(5):834-844). To determine whether high efflux capacity contributes to the survival of CX3CR1⁺ CD8⁺ T cells during chemotherapy, the efflux of a fluorescent anthracycline (doxorubicin) was measured in human primary CD8⁺ T cells isolated from healthy donors. The efflux of doxorubicin increased overtime in CX3CR1 CD8⁺ T cells (FIGS. 3A and 3B). As a consequence of efflux of the cytotoxic drug, fewer CX3CR1⁺ CD8⁺ T cells (efflux cells) than CX3CR1⁻ CD8⁺ T cells (non-efflux cells) underwent apoptosis (FIG. 3C). Since ABC-superfamily multidrug efflux proteins have been shown to contribute to chemoresistance in malignant cells (Gottesman et al., Nat Rev Cancer, 2002, 2(1):48-58), the expression of ABCB1 by CX3CR1⁺ cells was examined. Greater expression of ABCB1 was observed in CX3CR1⁺ CD8⁺ T cells than in CX3CR1⁻ CD8⁺ T cells (FIG. 3D). In addition, CX3CR1⁺ ABCB1⁺ double positive (DP) cells effluxed more Rh123 (a dye for measurement of efflux mediated by the ABCB1 transporter) than CX3CR1⁻ ABCB1⁻ double negative (DN) cells (FIG. 4), suggesting that ABCB1 may be a key transporter used by CX3CR1⁺ CD8⁺ T cells for drug efflux. To determine the role of ABCB1 in T cell drug efflux, studies were carried out to examine whether the efflux of doxorubicin could be blocked by PGP4008, a specific ABCB1 transporter inhibitor (Schinkel and Jonker, Adv Drug Deliv Rev, 2003, 55(1):3-29; and Walter et al., Blood, 2004, 103(11):4276-4284). The results, shown in FIG. 3E, show that PGP4008 at doses of 5-10 μM significantly suppressed efflux of doxorubicin by CX3CR1⁺ CD8⁺ T cells. Thus, the addition of PGP4008 increased apoptosis of CX3CR1⁺ CD8⁺ T cells in culture (FIG. 3F), suggesting that ABCB1 contributes to the survival of T cells during chemotherapy.

Since the pharmacodynamics of doxorubicin may not be able to exactly reflect the efflux of carboplatin and paclitaxel (CP), and these drugs cannot be directly tracked due to lack of fluorescent capability, the impact of the drug transporter inhibitor on the function of T cells in the presence of CP was examined to determine whether T cell function might be dampened due to the reduced ability of T cells to efflux CP. ABCB1 transporter inhibitor (PGP4008) was incubated with resting or activated human primary CD8⁺ T cells in vitro in the presence of CP. T cell function was measured by based pm degranulation (CD107a expression) and intracellular IFN-γ production. PGP4008 significantly inhibited the function of CX3CR1⁺ CD8⁺ T cells in the presence of CP (FIG. 3G), but not in the absence of CP (FIG. 3H). These results suggested that the drug efflux ability of CX3CR1⁺ CD8⁺ T cells via the ABCB1 transporter is required for endurance of chemotherapy and retention of function.

### Example 6—CX3CR1⁺ CD8⁺ T Cells Increased in Tumors after Effective CIT

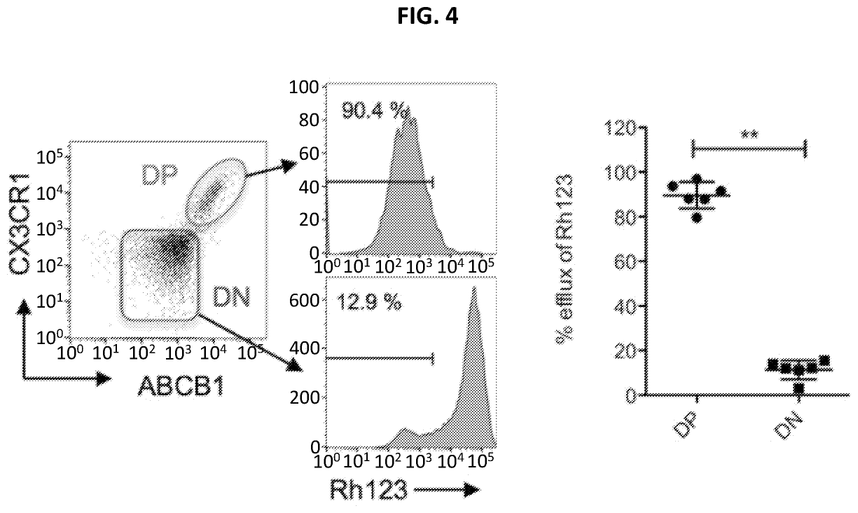

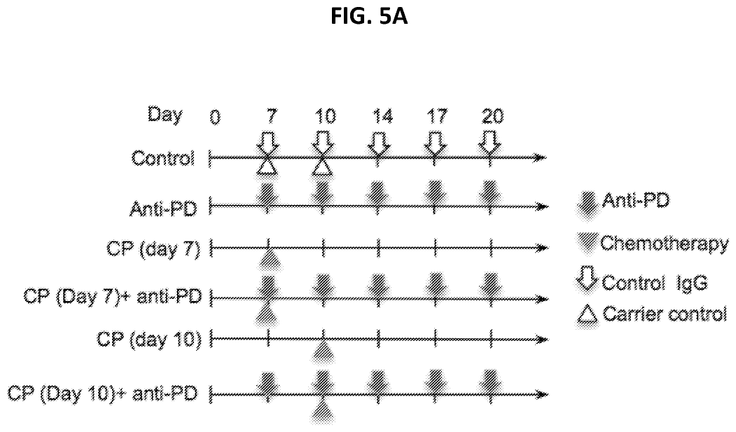

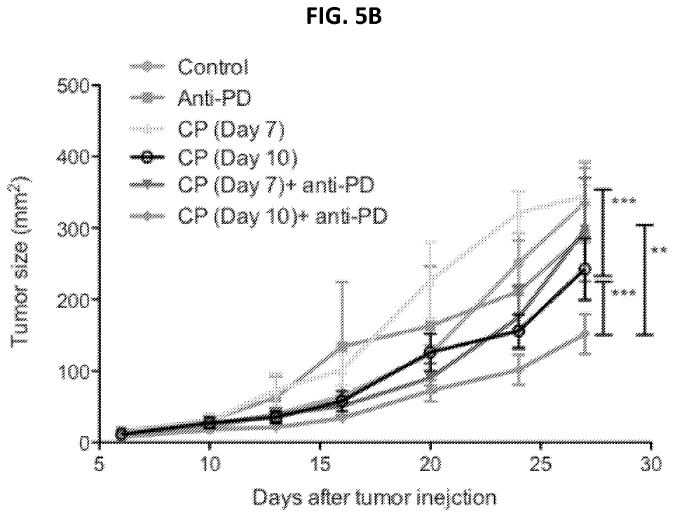

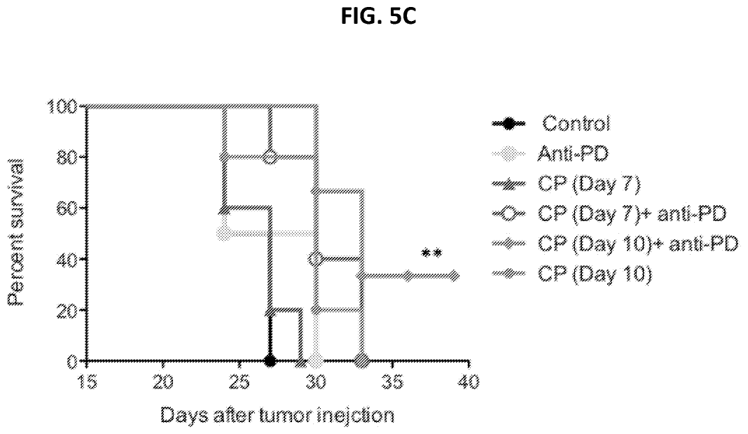

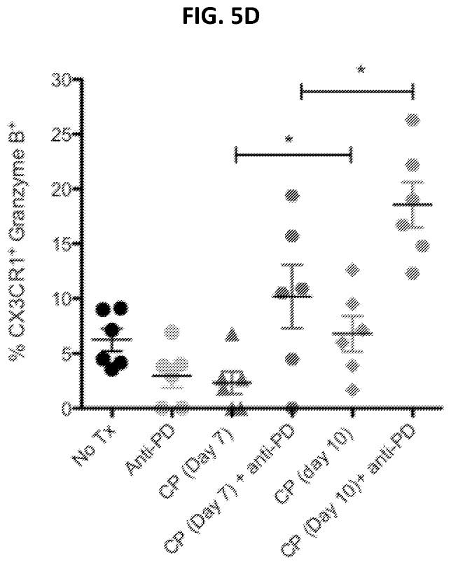

To examine whether the frequency of CX3CR1⁺ Granzyme B⁺ CD8⁺ T cells would reflect the therapeutic effects of the CIT, two schedules of CIT were designed, according to the two phases of T cell responses to tumors in an animal model (Liu et al., supra; and Pulko et al. J Immunol, 2011, 187(11):5606-5614). In this model, the frequency of tumor antigen specific effector CD8⁺ T cells peaked at day 10-14 post tumor inoculation within tumor tissues. According to the kinetics of T cell responses within tumors, the expansion phase was defined as days 7-9 and the effector phase was defined as days 10-14 of the antitumor responses. Anti-PD-1/L1 therapy was given to cover the expansion and effector phases according to the dynamic expression of PD-1 (Pulko et al., supra). Chemotherapy (CP) was given at either phase in order to evaluate its impact on T cell responses (FIG. 5A). The addition of CP on day 10 (effector phase) but not on day 7 (expansion phase) significantly suppressed the tumor growth of B16F10 mouse melanoma cells, in combination with anti-PD/L1 therapy (FIG. 5B), and prolonged the survival of treated mice (FIG. 5C). Accordingly, the frequency of CX3CR1⁺ Granzyme B⁺ effector CD8⁺ T cells had the highest increase in the group treated with CP plus anti-PD-1/L1 on day 10, compared to groups treated with either CP alone or with anti-PD-/L1 on day 7 (FIG. 5C). Of note, the frequency of CX3CR1⁺ Granzyme B⁺ CD8⁺ T cells was higher in mice treated with CP on day 10 than on day 7 even without combination with anti-PD/L1 (FIG. 5D), suggesting that the timing of chemotherapy may be critical to preserve CX3CR1⁺ Granzyme B⁺ CD8⁺ T cells that can be further improved by anti-PD therapy. In line with PD-1 blockade prior to chemotherapy, tumor growth in PD-1 knockout mice also was significantly suppressed by chemotherapy (CP), compared to wild type mice (FIG. 5E).

### Example 7—CX3CR1 is Required for CD8⁺ CTL to Reject Tumors During CIT

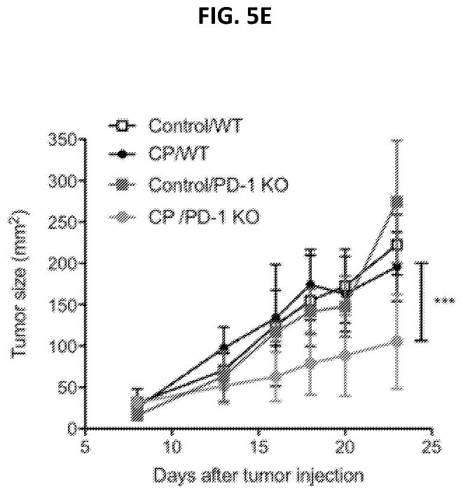

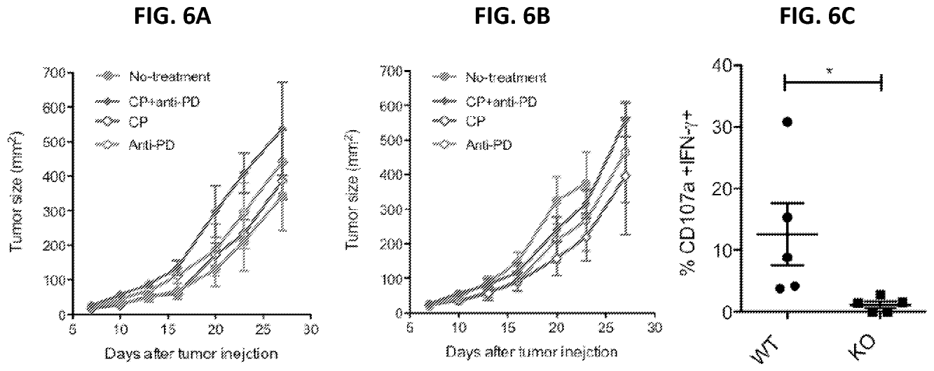

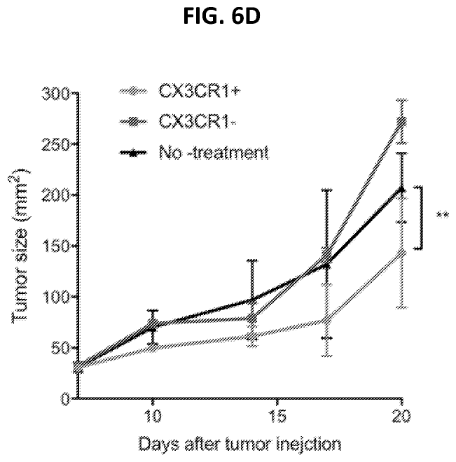

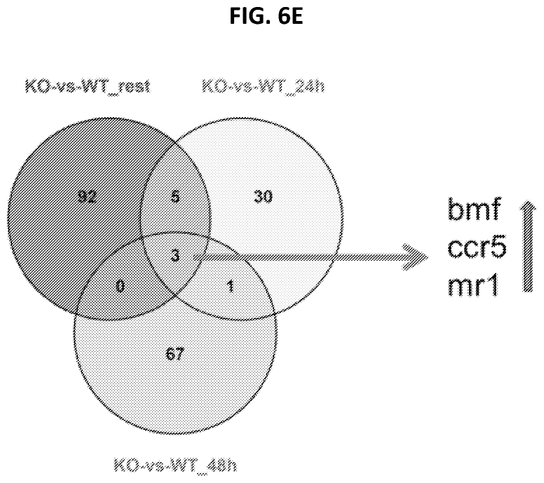

Since CX3CR1 is a chemokine receptor that is critical for accumulation of T cells at tumor sites (Kee et al., Mol Cin Oncol, 2013, 1(1):35-40), studies were conducted to examine whether the expression of CX3CR1 is required to mediate antitumor activity. Tumor cells were grown in CX3CR1 KO mice, followed by treatment with CIT (Day 10 CP plus anti-PD-1/L1). In contrast to wild type mice, the CIT did not suppress tumor growth in CX3CR1 KO mice (FIGS. 6A and 6B). In addition, the frequency of CD107a⁺ IFN-γ⁺ effector CD8⁺ T cells within tumors was significantly decreased in CX3CR1 KO mice as compared to wild type mice (FIG. 6C).

To address whether the CD8⁺ T cells specifically require CX3CR1 to mediate antitumor function, adoptive transfer of activated OT-1 CD8⁺ T cells was performed for treatment of a B16-OVA tumor model. The transfer of CX3CR1⁺ (but not CX3CR1⁻) CD8⁺ T cells significantly suppressed tumor growth (FIG. 6D), suggesting that CX3CR1 expression is critical for CD8⁺ CTL to mediate tumor rejection. To further examine the role of CX3CR1 in CD8⁺ T cells, the gene transcriptome was compared between wild type and CX3CR1 KO CD8⁺ T cells at resting or activated stages. As shown in FIG. 6E, expression of three genes (bmf, ccr5, and mr1) was consistently increased in CX3CR1 KO CD8⁺ T cells, regardless to their activation status. The bmf gene encodes a protein (Bcl-2 modifying factor) that functions as an apoptotic activator (Shao and Aplin, Cell Death Dis, 2012, 3:e253), and CCR5 has been reported to induce T cell apoptosis (Mellado et al., Curr Biol, 2001, 11(9):691-696; and Murooka et al., J Biol Chem, 2006, 281(35):25184-25194). This suggested that CX3CR1 expression may be required for CD8⁺ T cell survival through suppression of the transcription of apoptotic molecules (bmf and ccr5).

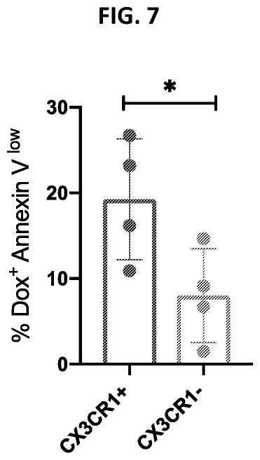

To determine the effect of chemotherapeutic treatment on survival of CX3CR1⁺ and CX3CR1⁻ CD8⁺ T cells, subsets of these cells were isolated, placed in 96 well plates at 2×10⁵ cells/well, and incubated with doxorubicin (Dox) at 0.5 μg/ml for 40 hours. After incubation, T cells were stained with annexin V. T cells affected by Dox were identified as Dox positive cells, and their survival was defined by low binding of annexin V. As shown in FIG. 7, the percentage of Dox⁺/annexin V low (live) cells was higher in the CX3CR1⁺ subset of CD8⁺ T cells than in the CX3CR1⁻ CD8⁺ subset.

Taken together, the studies described above indicate that CX3CR1 identifies a subset of tumor-reactive CD8⁺ T cells that can endure chemotherapy and are responsive to PD-1 blockade immunotherapy. The results also indicate that CX3CR1⁺ CD8⁺ T cells have at least two advantages allowing them to withstand the toxicity of 15 chemotherapy—drug efflux and downregulation of bmf and ccr5, and may play a key role in clinical responses to combined CIT.

### Example 8—Evaluating the Synergy of IL-15 and PD-1 Therapy in Treatment of Non-Responsive Tumors

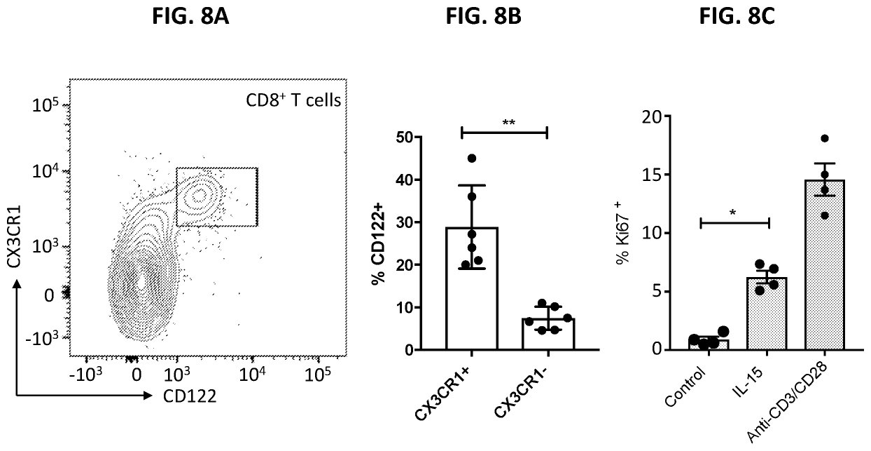

IL-15 has demonstrated antitumor function in preclinical models, especially as a IL-15/IL-15Ra complex that has increased accessibility to T cells in vivo (Stoklasek et al., J Immunol, 2006, 177:6072-6080). For at least a couple of reasons, IL-15 may improve anti-PD-1 therapy for non-responsive tumors. First, the transcription of CD122 (IL-2 receptor beta) was increased in CD11a^{high} CD8⁺ T cells in responders 25 compared to non-responders (FIG. 1B). In addition, CX3CR1⁺ CD8⁺ T cells exhibited increased CD122 expression and survival after IL-15 treatment. CX3CR1⁺ and CX3CR1⁻ CD8⁺ T cell subsets were incubated with PHA-L (5 μg/ml) for 48 hours, and the percentage of CD122⁺ cells was determined by flow cytometry (FIGS. 8A and 8B), revealing that % CD122⁺ was increased in CX3CR1⁺ CD8⁺ T cells as 30 compared to CX3CR1⁻ CD8⁺ T cells. Further, human PBMC were incubated with human IL-15 (10 ng/ml) or anti-CD3/CD28 beads for 48 hours, and proliferation of CX3CR1⁺ CD8⁺ T cells was assessed based on % Ki67⁺ cells. These studies showed that both treatments increased proliferation of the CX3CR1⁺ cells (FIG. 8C).

Because CD122 is a component of the IL-15 receptor, it is possible that increased sensitivity to IL-15 causes tumor-reactive CX3CR1⁺ Granzyme B⁺ CD8⁺ T cells to expand beyond the threshold and contribute to tumor rejection in responders, while in the non-responders the CX3CR1⁺ Granzyme B⁺ CD8⁺ T cells might have either lower CD122 expression or lower IL-15 production.

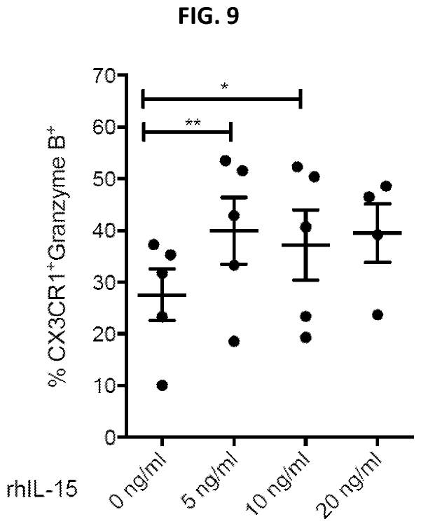

To test whether IL-15 directly contributes to the expansion of CX3CR1⁺ Granzyme B⁺ CD8⁺ T cells, human recombinant IL-15 was incubated for 24 hours with PBMC isolated from healthy human donors, followed by flow cytometry analysis of CX3CR1⁺ Granzyme B⁺ CD8⁺ T cells. IL-15 significantly increased the expansion of CX3CR1⁺ Granzyme B⁺ CD8⁺ T cells among other cells in the PBMC (FIG. 9). Thus, IL-15 may improve the efficacy of PD-1 ICI therapy by expanding CX3CR1⁺ Granzyme B⁺ CD8⁺ T cells that are capable of rejecting tumors.

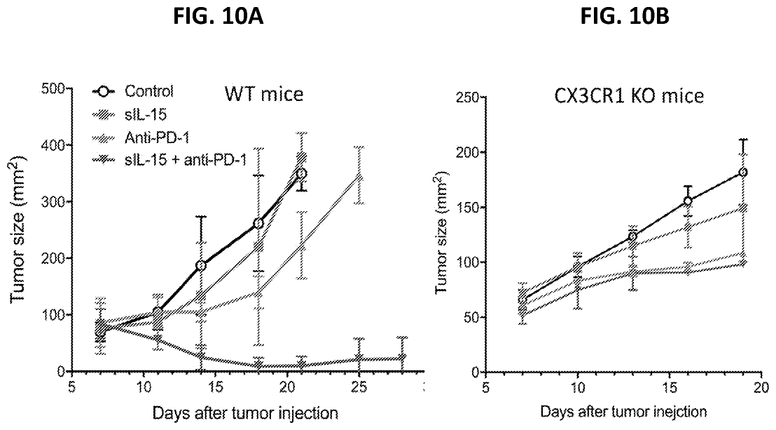

To address the mediators of IL-15 in context of its antitumor function, i.t. injection of IL-15/IL-15Ra complex in combination with i.p. injection of PD-1 antibody was evaluated for treatment of B16-OVA melanomas. Although IL-15 (at the experimental dose) alone did not suppress the growth of B16-OVA tumors, and anti-PD-1 alone only partially delayed the tumor growth, the combination of IL-15 and PD-1 antibody significantly suppressed tumor growth (FIG. 10A). To test the role of CX3CR1 expression, B16-OVA melanoma tumors growing in CX3CR1 KO mice were treated with IL-15 and/or PD-1 ICI following the same treatment protocol as in wild type mice. Strikingly, the synergistic effects of IL-15 and PD-1 ICI lost their therapeutic effects in suppression of tumor growth, compared to WT mice (FIG. 10B). Interestingly, the delayed tumor growth induced by PD-1 ICI therapy was not significantly affected by the lack of CX3CR1.

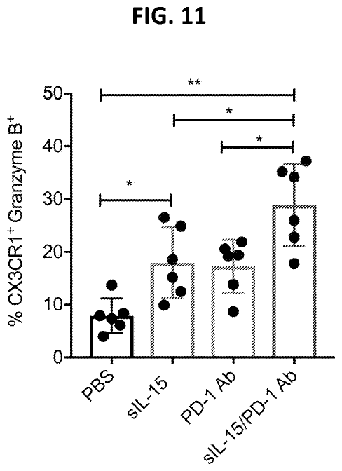

In additional studies, the effect of IL-15 and anti-PD-1 on CX3CR1⁺ effector T cells in tumor tissue was examined. B16-OVA melanomas were treated by i.t. injection of anti-PD-1 antibody (G4, 20 μg), soluble IL-15 (sIL-15) complex (mIL-15: 0.1 mg plus IL-15Ra chain: 0.6 mg), or both, for 3 doses on days 7, 10, and 13. The percentage of CX3CR1⁺ Granzyme B⁺ cells among CD11a⁺CD8⁺ TILs was determined on day 10 after tumor injection, which was 3 days after one dose of the various reagents. As shown in FIG. 11, the combined treatment led to the greatest increase in CX3CR1⁺ effector T cells.

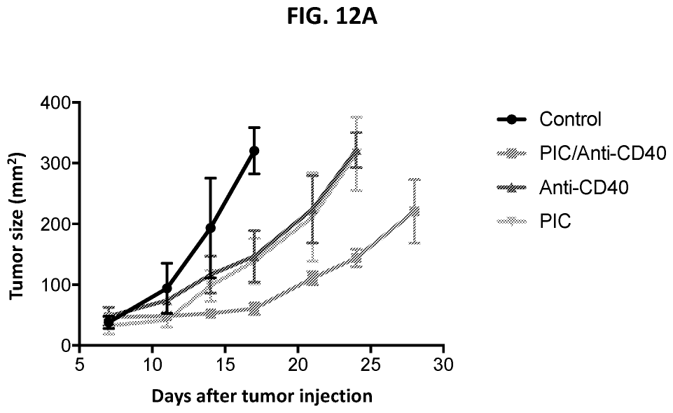

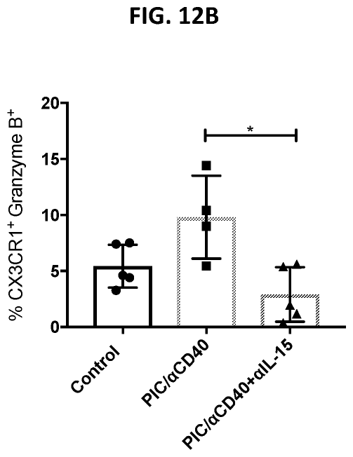

IL-15 blockade decreased CX3CR1⁺ effector cells in tumor tissues. B16-OVA tumors were treated with poly IC (PIC) and/or anti-CD40, which demonstrated antitumor activity (FIG. 12A) and induced CX3CR1⁺ effector CD8⁺ T cells (FIG. 12B; TILs analyzed on day 11). Peritumoral injection of an anti-IL-15 antibody on days 7, 8, and 9 after tumor injection abolished the increase in CX3CR1⁺ effector CD8⁺ T cells that was induced by poly IC and anti-CD40 (FIG. 12B).

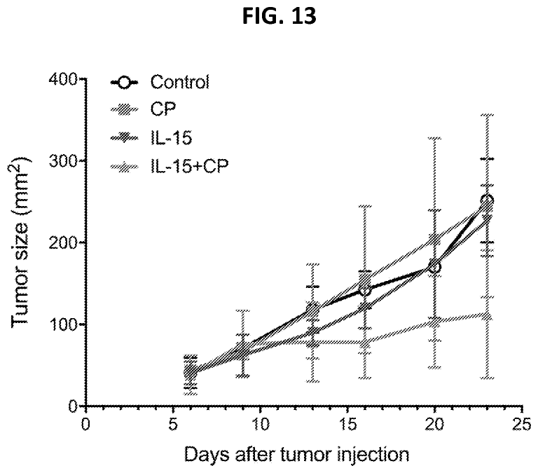

IL-15 also promoted the efficacy of chemotherapy. B16F10 mouse melanoma tumors were treated with carboplatin (40 μg/g) and paclitaxel (10 μg/g) by i.p. injection on day 10 after tumor injection (s.c. 5×10⁵ cells/mouse). Soluble IL-15 (sIL-15) complex (mIL-15: 0.1 mg plus IL-15Ra chain: 0.6 mg) was administered on days 7, 10, and 13 after tumor injection. As shown in FIG. 13, the combination of IL-15 and chemotherapy had the greatest effect on tumor size.

Collectively, these data suggested that PD-1 ICI can restore the antitumor function of pre-existing T cells, if the numbers of pre-existing T cells are not enough to compete rapid growing tumors, IL-15 is needed to expand additional antitumor effector T cells that are expressing CX3CR1 and have the ability move back to tumor site. Thus, in treatment of tumors that are non-responsive to PD-1 ICI therapy, IL-15 is a strong candidate for combination therapy.

Using this model, studies are conducted to determine whether the therapeutic effects of IL-15/PD-1 blockade are attributed to the increase in CX3CR1⁺ Granzyme B⁺ CD8⁺ T cells within tumors or in secondary lymph nodes, and whether the presence of CX3CR1⁺ Granzyme B⁺ CD8⁺ T cells would prevent treated mice from second challenges of same tumors. According to the treatment timing of FIG. 5A, TILs along with immune cells are isolated from draining lymph nodes and spleen on day 15 after last treatment of IL-15 or PD-1 antibody, or both. The % CX3CR1⁺ Granzyme B⁺ CD8⁺ T cells and their antitumor CTL function are examined by flow cytometry. The CTL function (degranulation/CD107a expression), proliferation (Ki67 expression), and cytokine production (IFN-γ and TNF-α) are measured after ex vivo brief stimulation with or without surrogate tumor antigen-OVA peptide as described elsewhere (Dronca et al., 2016, JCI Insight 1:e86014).

To test whether the synergy of IL-15 and PD-1 blockade in treatment of non-responsive tumors also is dependent on the presence of CX3CR1⁺ CD8⁺ T cells, tumor models are used (B16F10, LLC) in WT and CX3CR1 KO mice following the same treatment schedule as in FIG. 5A. The sizes of tumors, the phenotype (T cell activation markers, apoptosis, and proliferation) and function (CD107a and IFN-γ) of tumor-reactive CX3CR1 CD11a^{high} CD8⁺ T cells are measured and compared at 2-3 days after final treatments.

To more specifically address the role of CX3CR1⁺ CD8⁺ T cells in mediating the antitumor function of IL-15, studies are conducted to test whether transfer of IL-15 expanded CX3CR1⁺ Granzyme B⁺ CD8⁺ T cells can be used with PD-1 ICI to treat non-responsive tumors. Since IL-15 can selectively expand human CX3CR1⁺ Granzyme B⁺ CD8⁺ T cells in vitro (FIG. 9), the optimal dose and culture time of mouse IL-15 for expansion of mouse CX3CR1⁺ Granzyme B⁺ CD8⁺ T cells in vitro is determined. OT-1 CD8 T cells are used as a model because the transfer of CX3CR1⁺ CD8⁺ T cells activated with OVA antigen have the ability to suppress tumor growth (FIG. 6D). Before T cell transfer, the antitumor function of expanded CX3CR1⁺ Granzyme B⁺ CD8⁺ T cells is measured by flow cytometry after re-stimulation with OVA peptide for CD107a and cytokine production. The antigen-specific killing of tumor cells is determined by incubation of sorted CX3CR1⁺ CD8⁺ T cells with EL4 target cells loaded with cognate antigen peptide (OVA) or control peptide. Tumor lysis is measured using CYTOTOX 96® Non-Radioactive Cytotoxicity Assay kit (Promega Corp.; Madison, Wis.). Once the tumors are established, about on day 7-9, they are injected with 1×10⁶ sorted CX3CR1⁺ CD8⁺ T cells, followed by 5 doses of PD-1 antibody injection as in FIGS. 9, 10A, and 10B. In addition to B16-OVA tumor models, IL-15 is used to expand TILs isolated from B16F10, RENCA, and LLC tumors in order to expand tumor-reactive T cells, and to treat respective tumors in vivo in combination with PD-1 antibody.

### Example 9—Bim⁺ CD8⁺ T Cells Increased in Patients with Metastatic Melanoma

PD-1 blockade aims to block the engagement of PD-1 with its ligand PD-L1 in order to restore or enhance T cell function and survival (Dong et al., Nat Med, 2002, 8:793-800; and Iwai et al., Proc Natl Acad Sci USA, 2002, 99:12293-12297). Since none of the molecules in the PD-1 signaling pathway had previously been used to monitor the effects of PD-1 blockade in T cells, signaling molecules in the PD-1/PD-L1 pathway were investigated. These studies revealed that PD-L1 stimulates Bim up-regulation in activated CD8⁺ T cells as a mechanism for T cell apoptosis (Gibbons et al., Oncoimmunology, 2012, 1:1061-1073), and anti-PD-1 antibodies blocked the Bim up-regulation induced by PD-L1 protein or PD-L1 positive tumors in vitro and in vivo.

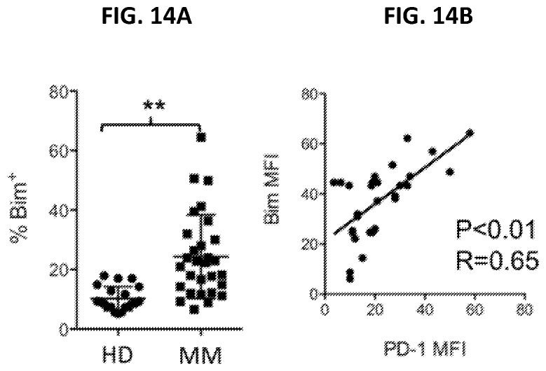

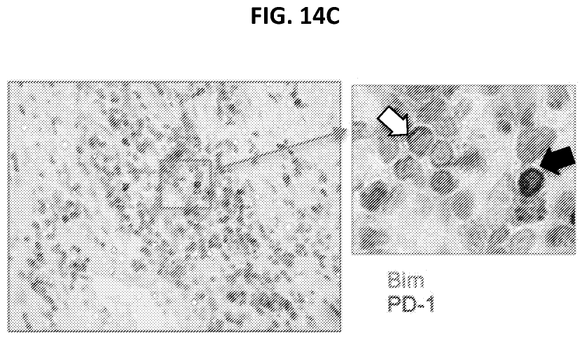

Further studies were conducted to examine and compare the frequency of Bim⁺ cells among circulating CD11a^{high} CD8⁺ T cells, since this population of T cells is enriched with tumor-reactive T cells (Liu et al., Oncoimmunology, 2013, 2:e23972). As shown in FIG. 14A, the percentages of Bim⁺ cells among CD11a^{high} CD8⁺ T cells (referred to as % Bim⁺ CD8⁺ T cells in the following sections) was 2.4-fold higher in the peripheral blood of patients with metastatic melanoma than in healthy donors (P<0.01). In addition, Bim levels (MFI) were positively correlated with PD-1 levels among CD11a^{high} CD8⁺ T cells of cancer patients (FIG. 14B, P<0.01; Duraiswamy et al., J Immunol, 2011, 186:4200-4212). Interestingly, some (but not all) PD-1 positive TILs expressed Bim within melanoma tissues (FIG. 14C), implying a functional diversity of PD-1⁺ T cells with respect to their engagement with ligands in the tumor microenvironment. These results indicated that PD-L1 contributes to Bim up-regulation in PD-1⁺ CD8⁺ T cells, which can be blocked by anti-PD-1 antibodies. Therefore, measurement of the frequency of Bim⁺ CD8⁺ T cells can be used to the degree to which PD-1 signals have been blocked in cancer patients.

### Example 10—PD-1 Blockade Decreased Bim⁺ CD8⁺ T Cells in Responders after PD-1 ICI Therapy

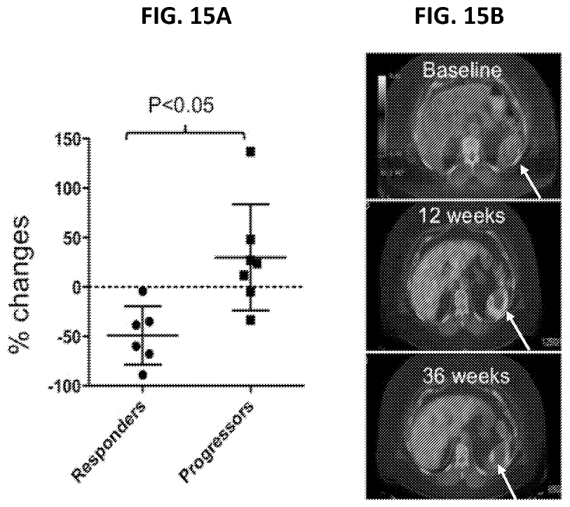

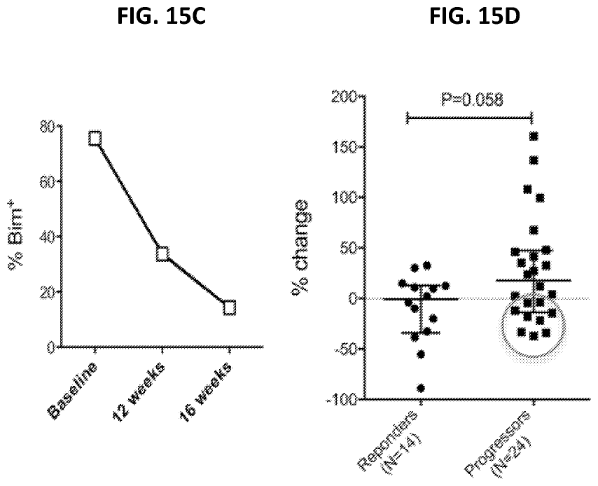

To determine whether the frequency of Bim⁺ CD8⁺ T cells would decrease in responders after PD-1 ICI therapy, the % Bim⁺ CD8⁺ T cells was examined and compared between responders and non-responders in a small cohort of patients with metastatic melanoma, 12 weeks after anti-PD-1 (pembrolizumab) therapy. Interestingly, it was observed that the % Bim⁺ CD8⁺ T cells significantly decreased in responders compared to non-responders at 12 weeks (FIG. 15A). Of note, in one patient, although a dramatic decrease in % Bim⁺ CD8⁺ T cells was observed at 12 weeks, the PET scan showed a “swelling lesion” in the spleen (arrow, FIG. 15B, middle) suggesting disease progression. However, a follow-up PET scan eventually confirmed a shrinking PET-avid lesion at the same site at 36 weeks (FIG. 15B, bottom), along with a further decline in the % Bim⁺ CD8⁺ T cells at 16 weeks after PD-1 therapy (FIG. 15C). These results suggested that a change in % Bim⁺ CD8⁺ T cells would be a more sensitive reflection of how a patient's immune system is responding to PD-1 ICI therapy, which cannot be directly evaluated by current imaging technology (CT or PEY scans).

Additional studies were carried out to validate this observation in another cohort of patients with metastatic melanoma. Most of the second cohort received PD-1 ICI therapy as first line therapy. Based on patents with clear clinical outcomes, the changes in % Bim⁺ CD8⁺ T cells at 12 weeks after PD-1 therapy were examined and compared in complete responders and in non-responders (with disease progression). Interestingly, although most of responders demonstrated a decrease in % Bim⁺ CD8⁺ T cells after PD-1 ICI therapy, some non-responders (about 40%) also had a decrease in % Bim⁺ CD8⁺ T cells after PD-1 ICI therapy (FIG. 15D, circled area). While the difference between the two cohorts of patients could be due to their previous treatments (the first cohort were in clinical trials while the second was treated with standard therapy), this new observation is important because it is the first evidence to indicate that PD-1 blockade actually works in non-responders, at least at T cell Bim levels. This new information is important for the design of new combined therapy to improve the efficacy of PD-1 ICI in this group of patients. Taken together, these results suggested that clinical responders clearly have a decrease in % Bim⁺ CD8⁺ T cells after PD-1 ICI therapy, but a mere decrease in % Bim⁺ CD8⁺ T cells may not always secure a clinical response or disease control.

### Example 11—Correlating Changes in Bim⁺ CD8⁺ T Cells and CX3CR1⁺ CD8⁺ T Cells after PD-1 Therapy

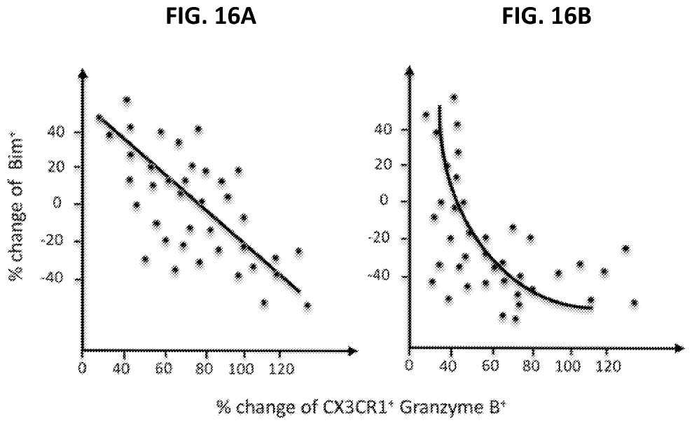

The findings discussed in the Examples above indicate a negative correlation between decreased % Bim⁺ CD8⁺ T cells and increased CX3CR1⁺ Granzyme B⁺ CD8⁺ T cells in melanoma patients after PD-1 ICI therapy, which would either follow a liner relationship or a curvilinear relationship (FIGS. 16A and 16B). Using Pearson correlation analysis, this hypothesis is tested, and the results are presented as the correlation coefficient (or “r”) along with statistical significance (P value).

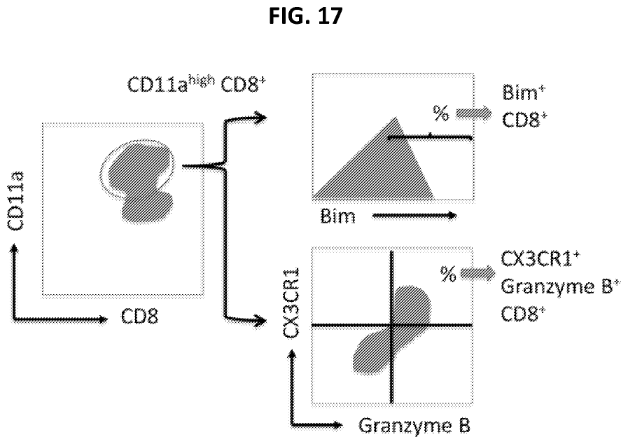

In particular, a prospective bio-specimen collection study is performed in a larger group of male and female patients with metastatic melanoma (about 100 people). This expansion allows a correlation of changes in Bim⁺ and CX3CR1⁺ Granzyme B⁺ CD8⁺ T cells after PD-1 therapy to be established. Fresh peripheral blood samples (e.g., 60 ml) are collected at baseline (prior to initiation of immunotherapy) and at 12 weeks after PD-1 ICI therapy. The tumor evaluation schedule is done per clinical practice every 6-12 weeks using both RECIST and irRC (Immune Related Response Criteria). Fresh PBMC are stained with antibodies to Bim, CX3CR1, Granzyme B, CD11a, CD8, CD3, and CD45 in the same tube to avoid variables in inter-tube staining of cell surface and intracellular molecules. Live CD45⁺CD3⁺ cells are gated followed by sub-gating of CD11a^{high} CD8⁺ T cells, as illustrated in FIG. 17. Among CD11a^{high} CD8⁺ T cells, the % Bim⁺ CD8⁺ and % CX3CR1⁺ Granzyme B⁺ CD8⁺ T cells is determined and presented as % Bim⁺ CD8⁺ T cells or % CX3CR1⁺ Granzyme B⁺ CD8⁺ T cells. Changes in Bim⁺ CD8⁺ T cells or CX3CR1⁺ Granzyme B⁺ CD8⁺ T cells at week 12 after PD-1 ICI therapy are calculated from baseline as reported elsewhere (Dronca et al., 2016, JCI Insight 1:e86014).

### Example 12—Collective Threshold of Changes in Bim⁺ CD8⁺ T Cells and CX3CR1⁺ CD8⁺ T Cells for Predicting Clinical Response to PD-1 ICI Therapy

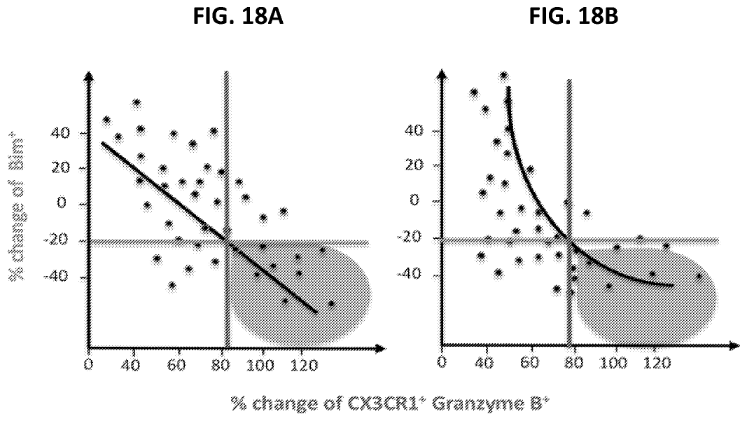

According to the studies on two cohorts of metastatic melanoma patients treated with anti-PD-1 antibody (FIGS. 15A-15D), the threshold of a decrease in Bim⁺ CD8⁺ T cells is estimated as >25% (range from −0.99 to −50%) in the % change of Bim⁺ CD8⁺ T cells from baseline for predicting an efficient PD-1 blockade-response in patients. This means that if the PD-1 blockade leads to at least a 25% reduction in Bim⁺ CD8⁺ T cells after PD-1 therapy, the PD-1 blockade is considered to be efficient. On the other hand, based on the above studies (FIGS. 1A-1E), the threshold of an increase in CX3CR1⁺ Granzyme B⁺ CD8⁺ T cells is estimated as >80% (range from 40-120%) in the % change of CX3CR1⁺ Granzyme B⁺ CD8⁺ T cells from baseline for predicting an efficient expansion of effector cells capable of rejecting tumors in cancer patients. As diagramed in FIGS. 18A and 18B, it is hypothesized that in either a liner or a curvilinear relationship between the changes in % Bim⁺ CD8⁺ T cells and % CX3CR1⁺ Granzyme B⁺ CD8⁺ T cells, if a patient has a collective change of a decrease in Bim⁺ CD8⁺ T cells greater than 25% and an increase in CX3CR1⁺ Granzyme B⁺ CD8⁺ T cells greater than 80%, this patient is most likely among the responders to anti-PD-1 therapy (shaded area in FIGS. 18A and 18B).

### Example 13—Tumor-Reactivity of CX3CR1⁺ Granzyme B⁺ CD8⁺ T Cells as a PD-1 Therapy-Responsive Cellular Marker

Studies are conducted to show the tumor-reactivity of circulating CX3CR1⁺ Granzyme B⁺ CD8⁺ T cells, establishing these cells as a reliable cellular marker for PD-1 therapy responsiveness. To determine tumor antigen specificity, gp100, tyrosinase, and MART-1 pentamer (ProImmune, Pro5 MHC Class I Pentamers) staining is performed using CX3CR1⁺ Granzyme B⁺ CD8⁺ T cells isolated from HLA-A0201⁺ patients. Functionally, HLA-A0201⁺ patient PBMCs are stimulated with pooled melanoma antigen peptides, and IFN-γ production is measured in CX3CR1⁺ Granzyme B⁺ CD8⁺ T cells as described elsewhere (Dronca et al., 2016, JCI Insight 1:e86014; and Romero et al., J Immunol, 2007, 178:4112-4119). To determine disease-specific T cell responses for patients who are not HLA-A0201⁺, DNA is extracted from CX3CR1⁺ CD8⁺ T cells isolated from peripheral blood (using age and gender-matched healthy donors as controls), and analyzed using an ImmunoSeq multiplex PCR assay (Adaptive Biotechnologies), followed by sequencing TCR beta CDR3 to identify and quantify clones of the CX3CR1⁺ CD8⁺ T cell subset. Clonal frequency is calculated as the ratio of clonal abundance of all the productive TCR sequences normalized to the number of unique TCR sequences. Since the RNA-seq data showed an increase in TCRVα5 and TCRVβ4-2 among CD11a^{high} CD8⁺ T cells in responders after PD-1 therapy (FIG. 1B), both RT-PCR and flow cytometry are used to examine whether this TCR use might be shared by melanoma patients.

Peripheral blood provides a less invasive way to directly assess T cell phenotypes in cancer patients, but there are functional and phenotypic differences between T cells present at the tumor sites and in circulation. To show whether circulating Bim⁺ or CX3CR1⁺ CD8⁺ T cells share similar T cell clones with their counterparts in tumor tissues, tumor biopsies are obtained and analyzed. DNA is extracted from CX3CR1⁺ Granzyme B⁺ T cells (sorted by flow cytometry) from peripheral blood and tumor tissues (laser capture for CX3CR1⁺ Granzyme B⁺ as shown in FIG. 1E), analyzed by an ImmunoSeq multiplex PCR assay, and sequenced for TCR beta CDR3 to identify and quantify clones of each subset of CD8⁺ T cell between peripheral blood and tissues. The tumor-antigen specificity of CX3CR1⁺ CD8⁺ T cells is expected to be determined by pentamers in HLA-A0201⁺ patients, and the T cell clonality assay is expected to find a consistent clonality between CX3CR1⁺ CD8⁺ T cells in peripheral blood and in tumor tissues for both HLA-A0201⁺ and non-HLA-A0201 patients.

To assess T cell differentiation, proliferation, and function, CX3CR1⁺ and CX3CR1⁻ CD8⁺ T cells isolated from melanoma patients are examined and compared before and after PD-1 ICI therapy. The endogenous proliferation of CX3CR1^{+/-} CD8⁺ T cells is examined by intracellular staining for Ki67, since Ki67⁺ cells have been identified in tumor-reactive CD8⁺ T cells in responders to PD-1 IC therapy (Huang et al., Nature, 2017, 545:60-65; and Kamphorst et al., Proc Natl Acad Sci USA, 2017, 114:4993-4998). If CX3CR1⁺ CD8⁺ T cells have increased proliferation after PD-1 ICI therapy in responders, further studies are conducted to determine the cytokine that contributes to their proliferation. To that end, CX3CR1^{+/-} CD8⁺ T cells are labeled with CFSE (an intracellular dye for cell division), and cultured with graded concentration of IL-2, IL-7, or IL-15 for 11 days. If spontaneous proliferation is not observed by day 5, the cells are removed to new culture wells containing anti-CD3/CD28 beads to initiate T cell proliferation with fresh cytokines. After incubation, the proportion of proliferative cells (CFSE dilution) between these two subsets is measured. In addition, studies are conduced to confirm whether cytokine receptor expression is different between CX3CR1⁺ CD8⁺ T cells and CX3CR1⁻ CD8⁺ T cells, or between responders ad non-responders, since transcription of CD122 (IL-2/IL-15Rβ) was increased in responders as compared to non-responders after PD-1 ICI (FIG. 1B).

CTL function (CD107a, Granzyme B, and perforin) and intracellular production of IFN-γ, TNF-α and IL-2 are examined ex vivo. To determine tumor antigen-induced function, PBMC are stimulated with pooled melanoma antigen peptides, and IFN-γ production is measured in CX3CR1^{+/-} CD8⁺ T cells as described elsewhere (Dronca et al., JCI Insight, 2016, 1: e86014; and Romero et al., J Immunol, 2007, 178:4112-4119). PBMC from patients who are not HLA-A0201⁺ are stimulated with anti-CD3/CD28 beads to trigger their CTL function.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method comprising:
measuring the percentage of CX3CR1⁺ cells and the percentage of Bim⁺ cells within a first population of CD8⁺ T cells obtained from a subject having a tumor, wherein said first population of CD8⁺ T cells was obtained prior to treatment of said subject with PD-1 blockade therapy;
measuring the percentage of CX3CR1⁺ cells and the percentage of Bim⁺ cells within a second population of CD8⁺ T cells obtained from the subject, wherein said second population of CD8⁺ T cells was obtained after treatment of the subject with PD-1 blockade therapy;
identifying said subject as having a percentage of CX3CR1⁺ cells within said second population that is increased by at least a predetermined CX3CR1⁺ threshold relative to said percentage of CX3CR1⁺ cells within said first population and as having a percentage of Bim⁺ cells within said second population that is decreased by at least a predetermined Bim⁺ threshold relative to said percentage of Bim⁺ cells within said first population; and
treating said subject with a therapy to increase tumor immunogenicity.

2-3. (canceled)

4. The method of claim 1, wherein said predetermined CX3CR1⁺ threshold is an increase of at least 80% and said predetermined Bim⁺ threshold is a decrease of at least 20%.

5. The method of claim 1, wherein said first and second populations of CD8⁺ T cells are from the peripheral blood of said subject, or wherein said first and second populations of CD8⁺ T cells are from said tumor.

6. (canceled)

7. The method of claim 1, wherein said subject is a human.

8. The method of claim 1, wherein said tumor contains metastatic melanoma cells, gastrointestinal cancer cells, genitourinary cancer cells, non-small lung cancer cells, or breast cancer cells.

9. (canceled)

10. The method of claim 1, wherein said therapy to increase tumor immunogenicity comprises radiation.

11. The method of claim 1, comprising measuring the percentage of CX3CR1⁺ Granzyme B⁺ cells within said first and second populations.

12. A method comprising:
measuring the percentage of CX3CR1⁺ cells and the percentage of Bim⁺ cells within a first population of CD8⁺ T cells obtained from a subject having a tumor, wherein said first population of CD8⁺ T cells was obtained prior to treatment of said subject with PD-1 blockade therapy;
measuring the percentage of CX3CR1⁺ cells and the percentage of Bim⁺ cells within a second population of CD8⁺ T cells obtained from said subject, wherein said second population of CD8⁺ T cells was obtained after treatment of said subject with PD-1 blockade therapy;
identifying said subject as having a percentage of CX3CR1⁺ cells within said second population that is increased by less than a predetermined CX3CR1⁺ threshold relative to said percentage of CX3CR1⁺ cells within said first population and as having a percentage of Bim⁺ cells within said second population that is decreased by at least a predetermined Bim⁺ threshold relative to said percentage of Bim⁺ cells within said first population; and
treating said subject with cytokine therapy combined with PD-1 blockade therapy.

13-14. (canceled)

15. The method of claim 12, wherein said predetermined CX3CR1⁺ threshold is an increase of at least 80% and said predetermined Bim⁺ threshold is a decrease of at least 20%.

16. The method of claim 12, wherein said first and second populations of CD8⁺ T cells are from the peripheral blood of said subject, or wherein said first and second populations of CD8⁺ T cells are from said tumor.

17. (canceled)

18. The method of claim 12, wherein said subject is a human.

19. The method of claim 12, wherein said tumor contains metastatic melanoma cells, gastrointestinal cancer cells, genitourinary cancer cells, non-small lung cancer cells, or breast cancer cells.

20. (canceled)

21. The method of claim 12, wherein said cytokine therapy comprises treatment with IL-15.

22. The method of claim 12, comprising measuring the percentage of CX3CR1⁺ Granzyme B⁺ cells within said first and second populations.

23. A method comprising:
measuring the percentage of CX3CR1⁺ cells and the percentage of Bim⁺ cells within a first population of CD8⁺ T cells obtained from a subject having a tumor, wherein said first population of CD8⁺ T cells was obtained prior to treatment of said subject with PD-1 blockade therapy;
measuring the percentage of CX3CR1⁺ cells and the percentage of Bim⁺ cells within a second population of CD8⁺ T cells obtained from said subject, wherein said second population of CD8⁺ T cells was obtained after treatment of said subject with PD-1 blockade therapy;
identifying said subject as having a percentage of CX3CR1⁺ cells within said second population that is increased by at least a predetermined CX3CR1⁺ threshold relative to said percentage of CX3CR1⁺ cells within said first population and as having a percentage of Bim⁺ cells within said second population that is increased, is unchanged, or is decreased by less than a predetermined Bim⁺ threshold relative to said percentage of Bim⁺ cells within said first population; and
treating said subject with combined chemo-immunotherapy (CIT).

24-25. (canceled)

26. The method of claim 23, wherein said predetermined CX3CR1⁺ threshold is an increase of at least 80% and said predetermined Bim⁺ threshold is a decrease of at least 20%.

27. The method of claim 23, wherein said first and second populations of CD8⁺ T cells are from the peripheral blood of said subject, or wherein said first and second populations of CD8⁺ T cells are from said tumor.

28. (canceled)

29. The method of claim 23, wherein said subject is a human.

30. The method of claim 23, wherein said tumor contains metastatic melanoma cells, gastrointestinal cancer cells, genitourinary cancer cells, non-small lung cancer cells, or breast cancer cells.

31. (canceled)

32. The method of claim 23, wherein said CIT comprises treatment with paclitaxel, carboplatin, and anti-PD-1 or anti-PD-L1 therapy.

33. The method of claim 23, comprising measuring the percentage of CX3CR1⁺ Granzyme B⁺ cells within said first and second populations.

34-72. (canceled)
